(19) United States
(12) Patent Application Publication
(10) Pub. No.: US 2026/0286430 A1
(43) Pub. Date: Sep. 24, 2026

(54) A METHOD FOR PROFILING OF MUTATIONS VIA GAP FILLING ON RNA AND A KIT FOR USE IN A METHOD FOR PROFILING OF MUTATIONS

(71) Applicant: VOXLBIO AB, Bandhagen (SE)

(72) Inventors: Mats NILSSON BERNITZ, Drottningholm (SE); Marco GRILLO, Sundbyberg (SE); Hao Zhe LEE, Solna (SE)

(21) Appl. No.: 19/168,113

(22) PCT Filed: Apr. 16, 2024
(86) PCT No.: PCT/SE2024/050359

§ 371 Date: Sep. 23, 2025

(30) Foreign Application Priority Data
Apr. 17, 2023 (SE) 2350459-0
Dec. 12, 2023 (SE) 2351415-1

(51) Int. Cl. C12Q 1/6869; C12N 15/10; C12Q 1/6806; C12Q 1/6841; C12Q 1/6853; C12Q 1/686

(52) U.S. Cl. CPC C12Q 1/6869; C12N 15/1096; C12Q 1/6806; C12Q 1/6841; C12Q 1/6853; C12Q 1/686

(57) ABSTRACT

The disclosure relates to a method for profiling of mutations, insertions, deletions and/or single or nucleotide variations in a RNA sample via gap filling, comprising the steps of: (a) providing an RNA sample comprising at least one nucleotide stretch of unknown identity to be identified, flanked by nucleotide stretches of known identity; (b) contacting the RNA sample with at least one DNA probe, such as at least two linear probes or at least one DNA padlock probe having a first and a second end under conditions and with reagents allowing hybridization, which first and second ends are designed to hybridize to the sequences of known identity flanking the at least one nucleotide stretch of unknown identity, thereby resulting in a gap between the hybridized first and second ends of the at least one DNA probe; (c) adding a polymerase with reverse transcriptase activity, and optionally nick translation activity, and optionally limited strand displacement, under conditions and with reagents allowing reverse transcription, thereby allowing reverse transcription of the at least one DNA probe from the first to the second end using the RNA sample as a template, so that the reverse transcribed part of the at least one DNA probe comprises the complementary sequence to the nucleotide stretch of unknown identity in the RNA sample; (d) adding a ligase that can use RNA as a splint molecule under conditions and with reagents allowing ligation, thereby allowing sealing of the at least one reverse transcribed DNA probe; (e) the probe containing the reverse transcribed nucleotide(s) can then be amplified; and (f) sequencing the at least one amplified circularized oligonucleotide, thereby revealing the identity of the at least one nucleotide stretch of unknown identity of the RNA sample. The disclosure further relates to a kit for use in a method for the profiling of mutations for both in situ and in vitro applications.

### TECHNICAL FIELD

The present disclosure relates to a method for profiling of mutations, insertions, deletions and/or single nucleotide variations and a kit for use in a method for profiling of mutations, insertions, deletions and/or single nucleotide variations directly on RNA. More specifically, the disclosure relates to a method for profiling of mutations and a kit for use in a method for profiling of mutations as defined in the introductory parts of the independent claims.

### BACKGROUND ART

The current state of the art method to perform capturing and sequencing of an unknown sequence flanked by known sequences (from now on defined as a “gap filling probe”) requires mRNA to be first transcribed to cDNA by reverse transcription (RT) Subsequently, a gap filling padlock probe or set linear probes are hybridised to the target cDNA and gap filling is performed on the in situ generated cDNA. Finally, the closed gap is ligated by a specific ligase. From published works, it is known that this process is strongly limited by the inefficiency of in situ RT as well as the need to post fix the generated cDNA with fixative also adversely affects the detection efficiency. A further bottleneck is added by the relative inefficiency of ligation to seal the filled sequence to the flanking probe(s) before their amplification (https://doi.org/10.1093/nar/gkx1206).

US20220042084A1 discusses using a gap filling padlock probe on either cDNA or mRNA. It focuses on optimization and use of the enzyme related to the RCA, where BST is used as DNA polymerase vs reverse transcriptase. Moreover, TTH DNA polymerase is considered as a reverse transcriptase for gap filling. EP4039822A1 discloses a method of combining targeted RNA or cDNA using padlock oligonucleotides. In both these references, reverse transcription and ligation are considered as two different steps. Also, US20210238662A1 discloses the use of BST polymerase as DNA polymerase for rolling circle amplification (RCA) applications instead of it as a reverse transcription enzyme.

Further, WO2013119827 discloses the use of TTH DNA polymerase as the RT and T4 DNA ligase for ligation.

There is no existing method or product in the market to profile mutations and insertions or deletions on mRNA both directly in situ, while preserving tissue morphology, nor in vitro. The current state of the art method involves first transcribing mRNA to cDNA in situ by reverse transcription, before performing a gap filling experiment on cDNA. Reverse transcription (RT) in situ is inefficient and the post fixation of cDNA in the intracellular matrix within biological samples after RT is further detrimental to detection efficiency. Moreover, using FFPE samples is inefficient due to the fragmented mRNA. Overall, the limited efficiency of this detection method strongly restricts its field of application. Attempts have been made to use mRNA directly as a template, thereby e.g., avoiding the extensive step of transcribing mRNA to cDNA. However, enzymes and conditions used have not been optimized and does not solve the problem of efficiently and reliably performing the gap filling, thereby providing in situ profiling results at the required level.

There is thus a need for improved methods and products aimed at both in vitro and in situ profiling of mutations and insertions/deletions on RNA directly.

### SUMMARY

It is an object of the present disclosure to mitigate, alleviate or eliminate one or more of the above-identified deficiencies and disadvantages in the prior art and solve at least one of the above-mentioned problems.

The present disclosure presents a method and a kit for profiling mutations and insertions or deletions directly on mRNA on biological samples, optionally in situ, while preserving tissue morphology.

According to a first aspect there is provided a method for profiling of mutations, insertions, deletions and/or single or nucleotide variations in an RNA sample, comprising the steps of:
(a) providing an RNA sample comprising at least one nucleotide stretch of unknown identity to be identified, flanked by nucleotide stretches of known identity;
(b) contacting the RNA sample with at least one DNA probe, such as at least two linear probes or at least one DNA padlock probe, having a first and a second end under conditions and with reagents allowing hybridization, which first and second ends are designed to hybridize to the sequences of known identity flanking the at least one nucleotide stretch of unknown identity, thereby resulting in a gap between the hybridized first and second ends of the at least one DNA probe;
(c) adding a polymerase with limited reverse transcriptase activity, and optionally nick translation activity, and optionally limited strand displacement, under conditions and with reagents allowing reverse transcription, thereby allowing reverse transcription of the at least one DNA probe from the first to the second end using the RNA sample as a template, so that the reverse transcribed part of the at least one DNA probe comprises the complementary sequence to the at least one nucleotide stretch of unknown identity in the RNA sample;
(d) adding either a ligase that can use RNA as a splint molecule under conditions and with reagents allowing ligation, or using chemical ligation such as click chemistry, thereby allowing sealing of the at least one reverse transcribed DNA probe in order to generate at least one linear or circularized oligonucleotide;
(e) amplifying the at least one linear or circularized oligonucleotide under conditions and with reagents allowing rolling circle amplification, thereby generating at least one amplified circularized oligonucleotide, or extracting the ligated probe from the RNA sample for downstream PCR amplification; and
(f) sequencing the at least one amplified oligonucleotide, thereby revealing the identity of the nucleotide stretch of unknown identity of the RNA sample.

Hereby, a method is presented which efficiently and reliably reveals the identity of a mutation, insertion, deletion or single nucleotide variation in an RNA sample. Especially, in situ profiling of mutations and insertions in line with the present disclosure is advantageous as one can map the location of these mutations back onto the tissue space, which can, for example, allow researchers to investigate the tumor microenvironment where different mutations reside within the tumor. Such information is important as it gives information with regards to e.g., cancer outcomes such as growth, progression and recurrence (see e.g., https://doi.org/10.1038/s41586-022-05425-2).

According to some embodiments, the stretch of unknown identity of the RNA sample comprises at least one nucleotide position to be profiled wherein the at least one nucleotide position to be profiled is an insertion, deletion and/or a single or multiple nucleotide variation in the RNA sample.

According to some embodiments, the RNA sample is an mRNA, rRNA or other non-coding RNA sample.

Thus, many types of RNA samples can be profiled by the present method.

According to some embodiments, at least one DNA probe is chosen from (i) at least one padlock probe, (ii) at least two linear probes that can be circularized by introducing a bridging probe and a ligase, (iii) at least one padlock probe or at least two linear probes that can be enzymatically ligated and can be released from the RNA sample, amplified and sequenced via NGS (next generation sequencing), or (iv) at least one padlock probe or at least two linear probes that can be chemically ligated via click reaction, thereby not being circularized and can be released from the RNA sample, amplified and sequenced via NGS.

Thus, the DNA probe(s) is/are designed to hybridize on each side of the nucleotide stretch of unknown identity to be identified, thereby forming a gap to be filled, and is/are designed to be either circularized and thereafter amplified in a rolling circle amplification reaction, or to be ligated enzymatically or chemically, before being released from the RNA sample, amplified and sequenced via NGS chemistries.

According to some embodiments, the first end of the at least one DNA probe is designed to hybridize to the RNA sample to the nucleotide stretch of known identity at the 3′ side of the nucleotide stretch of unknown identity, and the second end of the DNA probe is designed to hybridize to the RNA sample to the nucleotide stretch of known identity at the 5′ side of the nucleotide stretch of unknown identity.

Hence, the design of at least one DNA probe is essential, so that it hybridizes properly to the RNA sample.

According to some embodiments, the at least one DNA probe is either phosphorylated or unphosphorylated at the 5′ end. The only ligase available on the market capable of ligating DNA sequences splinted by an RNA molecule efficiently is PBCV1-ligase (commercially distributed as SplintR ligase). The activity of this ligase is only marginally inhibited by mismatches between the RNA template and the complementary DNA probes to ligate, being unable to discriminate between perfectly matched sequences and mismatched ones. SplintR can even ligate across gaps of variable length, depending on the abundance of the template RNA (in our experiments we were able to achieve significant ligations even across a 20 nt gap on very abundant targets). This spurious activity is inversely proportional to the size of the gap, with smaller gaps being spuriously ligated at higher frequencies.

In gap filling applications where the RT-gap filling step and the ligation steps are combined in a single reaction, this promiscuous activity of SplintR is potentially problematic: as the gap gets shorter during the gap filling reaction, the change of spurious ligation increases. In practical terms, in this type of experimental setting, the profiling of the gap sequence has a decreasing accuracy from 3′ to 5′. This is of course very problematic for small gaps (such as 0 or 1 nt gaps) as the rate of spurious ligation can be very high.

A strategy to mitigate this problem would be to chronologically separate the filling reaction from the ligation step, so to ensure that a high percentage of the gaps have been completely filled before sealing.

However, depending on the gap-filling enzyme of choice, this may not possible: Bst polymerase, for instance, has a nick-translation activity and, when incubated for long time, will progressively degrade the 5′ probe (or 5′ arm of the padlock), reducing its hybridization stability. This can be partially mitigated by the use of exo-nuclease resistant nucleotides in the 5′ probe, preventing excessive degradation via the nick-translation activity of Bst.

Another, non-exclusive, approach is to take advantage of the nick translation activity of Bst to make ligation conditional to the completion of the gap filling reaction, even when the two reactions are performed simultaneously. This is achieved by the use of non 5′Phosporylated padlock probes or 5′probes. These probes will carry a non-ligatable 5′-OH terminus. This prevents the probes to be ligated spuriously by the ligase, because a 5′-phosphate is necessary for ligation. Thus, when the gap filling is completed by a polymerase/reverse transcriptase with nick-translation activity, the 5′-OH terminus will be cleaved by the polymerase/reverse transcriptase, exposing the next base's phosphate.

In summary, a DNA polymerase with reverse transcription activity and having nick translation activity (such as BST Polymerase Full Length) may be used to expose a phosphorylated 5′ end of the at least one DNA probe and allow the sealing of the gap conditionally to its complete filling. Thus, this opens up an approach (1) for combining reverse transcription and ligation steps, and/or (2) for profiling short insertions and/or point mutations with little or no unspecific detection, and/or (3) for profiling longer insertions and/or mutations with a limited unspecific detection.

According to some embodiments, the lengths of the first and second ends of the at least one DNA probe that are designed to hybridize to the RNA sample to the nucleotide stretches of known identity are in the interval of 10-30 nucleotides.

Hereby, the at least one DNA probe is designed to hybridize properly to the RNA sample.

According to some embodiments, the gap between the first and second ends of the at least one DNA probe hybridized to the RNA sample, corresponding in length to the stretch of unknown identity to be identified, is in the interval of 0-20 nucleotides.

By using the technology of the present disclosure mutations, insertions and/or deletions of various lengths can be profiled. Moreover, by using a gap of 0 nucleotides (i.e., no gap) when using non-phosphorylated probes, false positives could be decreased as any non-specifically bound probe will have a zero chance of generating signal, compared to a non-zero chance from a phosphorylated probe and a mismatch tolerant ligase.

According to some embodiments, the polymerase with reverse transcriptase activity is chosen from the group comprising BST DNA polymerase (Full Length), TTH DNA Polymerase and DNA Polymerase I, as well as engineered mutant polymerases derived from the thermophilic bacterium Bacillus stearothermophilus, family A of DNA polymerases and other polymerases and reverse transcriptases exhibiting low strand displacement activity and/or good RT fidelity.

Hereby, examples of suitable enzymes for the step of in situ reverse transcription of the padlock probe using the RNA sample as a template are provided. Other polymerases exhibiting a low or limited strand displacement activity and/or good RT fidelity and engineered, mutant polymerases/reverse transcriptases could also be conceived for the purposes of the present disclosure, e.g., those that is able to accept modified dNTPs such as nucleotides with a 3′-azide group for click chemical ligation.

According to some embodiments, the polymerase with reverse transcriptase activity is a DNA polymerase with limited reverse transcription activity and nick translation activity that is derived from the thermophilic bacterium Bacillus stearothermophilus or family A of DNA polymerases.

According to some embodiments, the ligase is chosen from Splint R ligase, PBCV-1 DNA ligase and/or Chlorella virus DNA ligase.

Hereby, examples of suitable enzymes (RNA or DNA ligases) for the sealing of the reverse transcribed probe(s) in order to generate a linear/circularized oligonucleotide are provided.

The ligase is typically an RNA or DNA ligase being able to accept a DNA/RNA hybrid for ligation and which ligase has a high ligation activity. It should be noted that Splint R ligase, PBCV-1 DNA ligase and/or Chlorella virus DNA ligase are essentially the same enzyme (but with different names in different contexts).

According to some embodiments, the hybridized 5′ end of the DNA probe(s) in step (b) contains a 5′ alkyne group and after reverse transcription with nucleotides with a 3′-azide group, the nick between adjacent 5′-alkyne and 3′-azide group is to be chemically ligated via CuAAC click chemistry.

Hereby, the ligation can be performed via chemically via copper catalyzed azide alkyne cycloaddition (CuAAC) click reaction.

According to some embodiments, the steps of reverse transcription (step (c)) and ligation (step (d)) of the at least one DNA probe are combined, thereby allowing reverse transcription and sealing of the padlock probe within the same step.

According to some embodiments, the nick between the probe(s) is ligated chemically via click chemistry.

Hereby, the gap should be filled with nucleotides having a 3′-azide group, Hereby, a further simplified method is provided.

According to some embodiments, an additional step of nick translation is performed together with the reverse transcription and before the nick sealing with a ligase of step (d).

According to some embodiments, the DNA probe(s) provided in step (b) are non-phosphorylated, and the combination of steps (c) and (d) where the reverse transcription and nick translation is conditional for probe activation, before nick sealing can occur.

According to some embodiments, the reverse transcription is performed with 3′-azide nucleotides, before the nick sealing with click chemistry.

According to some embodiments, the DNA probe(s) comprises a 5′-hexynyl group and reverse transcription is performed with nucleotides conjugated with 3′-azide group. Upon incorporation of the azide labelled base, a copper catalyzed azide-alkyne cycloaddition (CuAAC) reaction can be performed, ligating and sealing the nick.

According to some embodiments, the DNA probe(s) comprises a 5′-hexynyl group and reverse transcription is performed with both deoxyribonucleotide triphosphates and nucleotides conjugated with 3′-azide group. Upon incorporation of the azide labelled base, a copper catalyzed azide-alkyne cycloaddition (CuAAC) reaction can be performed, ligating and sealing the nick.

In practice, the reagents and enzymes necessary for the reverse transcription, including the nick translation (if applicable), and the enzymatic ligation (nick sealing) may be added simultaneously, thereby performing the reactions in direct association with each other. For example, when using a non-phosphorylated 5′ end, as soon as the reverse transcriptase has filled the gap (if any) and has activated the non-phosphorylated 5′ end by cleaving off one nucleotide, the ligase can seal the nick. According to some embodiments, the DNA probe(s) provided in step (b) comprises at least one exo-nuclease resistant nucleotide close to the 5′ end, and wherein in step (c) (i) a polymerase with reverse transcriptase activity and nick translation activity, and (ii) a universal base primer, having a length corresponding to or being slightly shorter than the gap, are added, wherein the universal base primer binds to the stretch of unknown identity of the RNA sample and the polymerase thereafter nicks and converts the bases of the universal base primer to bases complementary to the stretch of unknown identity of the RNA sample, after which the gap is sealed.

According to some embodiments, the DNA probe(s) provided in step (b) comprises phosphorothioate bond or a LNA base close to the 5′ end, and in step (c) (i) a polymerase with reverse transcriptase activity and nick translation activity, and (ii) a universal base primer, having a length corresponding to or being slightly shorter than the gap, are added, wherein the universal base primer binds to the stretch of unknown identity of the RNA sample and the polymerase thereafter nicks and converts the bases of the universal base primer to bases complementary to the stretch of unknown identity of the RNA sample, after which the gap is sealed.

The DNA probe(s) can be one “padlock” probe, or two split probes, i.e., linear probes.

The term “gap-filled bases” refers to bases that represent a complementary sequence to the DNA/RNA template sequence to which they bind.

The term “universal bases” refers to bases that can base pair with all four standard bases in DNA (or correspondingly in RNA). Examples include 2′-Deoxylnosine and 2′-DeoxyNebularine. Also, for example nitroindole bases as per IDT (Integrated DNA Technologies) can be used (https://eu.idtdna.com/site/Catalog/Modifications/Product/140). See also Liang et al (RSC Adv., 2013,3, 14910-14928, “Universal base analogues and their applications in DNA sequencing technology”) for a review.

Typically, the universal bases are added as a universal base primer, having a length corresponding to the gap between the 3′ and 5′ end of the DNA probe(s), or wherein said universal base primer is slightly shorter than the gap. The purpose of the universal bases is to further stabilize probe hybridization to the target as compared to having just a gap. For this embodiment using universal bases, the same family of reverse transcriptase enzyme and ligase enzymes can be used as for other embodiments.

Hereby, the nick translation activity, when using a polymerase having such activity, can be controlled, and will avoid undesired displacement of the probe(s) thereby reducing capture efficiency.

In some embodiments, 1, 2, 3, 4, 5, 6, 7, 8, 9, 10, for example 3-7 and especially about 5 consecutive nucleotides at the 5′ end of the DNA probe comprise(s) phosphorothioate bonds.

For the technology to work, typically at least one normal base (not comprising a phosphorothioate bond) exists at the 5′ end for the reverse transcriptase to cleave/nick translate to expose a 5′-phosphate group for ligation. Therefore, the phosphorothioate bonds can typically be at closest 1 base away from the 5′ end. Further, the phosphorothioate bonds should be as close as possible to the ligation junction. Thus, in one embodiment, the phosphorothioate bonds are included at the 5′ end of the DNA probe starting from the second base from the 5′ end. In some embodiments, the PS bonds extends from the 2nd to about the 4^{th}, 5^{th}, 6^{th}, 7^{nd}, 8^{th}, 9^{th}, 10^{th} or 11^{th}, especially from the 2^{nd} to about the 6^{th} of the bases counted from the 5′ end of the DNA probe(s).

In some embodiments, 1, 2, 3, 4, 5, 6, 7, 8, 9, 10, for example 1-5 and especially about 1 consecutive nucleotides at the 5′ end of the DNA probe starting from the second base from the 5′ end comprise LNA nucleotide(s).

According to some embodiments, the phosphorothioate bonds and/or LNA nucleotide(s) are included at the 5′ end of the DNA probe starting from the second base from the 5′ end.

For the technology to work, typically at least one normal base (not comprising a LNA nucleotide) exists at the 5′ end for the reverse transcriptase to cleave/nick translate to expose a 5′ phosphate group for ligation. Therefore, the LNA nucleotide can typically be at closest 1 base away from the 5′ end. Further, the LNA nucleotide should be as close as possible to the ligation junction. Thus, in one embodiment, the LNA nucleotide is included at the 5′ end of the DNA probe starting from the second base from the 5′ end. In some embodiments, the LNA nucleotide extends from the 2^{nd} to about the 4^{th}, 5^{th}, 6^{th}, 7^{nd}, 8^{th}, 9^{th}, 10^{th} or 11^{th}, especially from the 2^{nd} to about the 6^{th} of the bases counted from the 5′ end of the DNA probe(s).

Hereby, an improved and novel probe design for gap filling on RNA or DNA to improve detection efficiency for controlled nick translation is provided:
1. Prevention of probe dissociation by uncontrolled nick translation by introducing phosphorothioate bonds at the 5′ end of the probe to control gap filling/nick translation.
2. Prevention of probe dissociation by uncontrolled nick translation by introducing LNA nucleotide(s) at the 5′ end of the probe to control gap filling/nick translation.
3. Introduction of universal bases across an unknown gap of sequence that would allow for improved probe hybridization stability and thus rely on nick translation activity to convert the universal bases into gap filled bases.

According to some embodiments, a plurality of stretches of unknown identity are profiled in the same experiment, wherein each stretch of unknown identity is flanked by stretches of known identity, and whereby, for each stretch of unknown identity, at least one DNA probe having a first and a second end is/are provided, which are designed to hybridize to the sequences of known identity flanking the nucleotide stretch of unknown identity, thereby allowing profiling of multiple mutations, insertions, deletions and/or single nucleotide variations.

According to some embodiments, wherein if the method is performed in situ, sequencing is performed in situ, such as by Sequencing by Synthesis (SBS), Sequencing by Ligation (SBL), Sequencing by Hybridisation (SBH), SOLiD sequencing or single base extension sequencing.

Hereby, the identity of the stretch of unknown identity of the RNA sample is revealed and profiled in situ.

According to some embodiments, wherein the method is performed in vitro, sequencing is performed via NGS sequencing post extraction of DNA probe(s) from the RNA sample.

According to some embodiments, wherein if the method is performed in situ, the probes, either pre or post amplified, are extracted from the RNA sample and processed for NGS sequencing.

Hereby, the identity of the stretch of unknown identity of the RNA sample is revealed and profiled via NGS.

According to some embodiments, the method is used for both in situ and in vitro applications of: mutation profiling, sequencing of insertion or deletions, CRISPR validation, lineage tracing, barcoding applications, miRNA profiling, profiling of unknown sequences in RNA in solution and/or mutation profiling for diagnostics applications.

The technology of the current disclosure can be used for a plurality of different applications.

According to a second aspect there is provided a kit for use in a method according to the first aspect for in-situ and/or in vitro profiling of mutations, insertions, deletions and/or single nucleotide variations in an RNA sample, wherein the RNA sample comprises at least one nucleotide stretch of unknown identity, flanked by nucleotide stretches of known identity, comprising:
(i) one or more DNA probes, chosen from (a) at least two linear probes or (b) one or more DNA padlock probes, having a first and a second end, which first end is designed to hybridize to the RNA sample to the at least one nucleotide stretch of known identity at the 3′ side of the nucleotide stretch of unknown identity, and the second end is designed to hybridize to the RNA sample to the nucleotide stretch of known identity at the 5′ side of the nucleotide stretch of unknown identity, optionally designed to be chemically ligated via click reaction;
(ii) a polymerase with reverse transcriptase activity and optionally limited strand displacement, optionally including necessary reagents and buffers;
(iii) a ligase that can use RNA as a splint molecule, optionally including necessary reagents and buffers;
(iv) optionally one or more amplification primers and a polymerase for linear or rolling circle amplification, and necessary reagents and buffers;
(v) optionally means for sequencing the amplification product;
(vi) optionally means for library preparation;
(vii) optionally universal bases; and
(viii) instructions for use.

Hereby, kits are provided for performing the method of the first aspect.

According to some embodiments, the polymerase with reverse transcriptase activity is chosen from the group comprising BST DNA polymerase (Full Length), TTH DNA Polymerase and DNA Polymerase I, as well as polymerases, such as mutant polymerases, derived from the thermophilic bacterium Bacillus stearothermophilus, family A of DNA polymerases and other polymerases and reverse transcriptases exhibiting low strand displacement activity.

According to some embodiments, the DNA ligase is chosen from Splint R ligase, PBCV-1 DNA ligase and/or Chlorella virus DNA ligase.

According to some embodiments, the polymerase with reverse transcriptase activity is chosen from the group comprising BST DNA polymerase, TTH DNA Polymerase and DNA Polymerase I, as well as polymerases exhibiting low strand displacement activity and 3′-azide nucleotides are added in the reaction mix for reverse transcription.

According to some embodiments, the kit further comprises means and reagents for performing the ligation via CuAAC click chemistry, thus ligating the 3′-azide and 5′-alkyne group at the nick junction of the DNA probe(s).

According to some embodiments, the at least one DNA probes are designed to exhibit a gap in the interval of 1-20 nucleotides between the first and second end upon hybridization to the RNA sample and/or wherein the at least one DNA probes are either phosphorylated or unphosphorylated at the 5′ end, and/or wherein the lengths of the first and second ends of the at least one DNA probe that are designed to hybridize to the RNA sample to the nucleotide stretches of known identity are in the interval of 10-30 nucleotides.

According to some embodiments, the at least one DNA probe(s) are designed to exhibit a gap in the interval of 1-20 nucleotides between the first and second end upon hybridization to the RNA sample and/or wherein the at least one hybridized DNA probe has an alkyne functional group at the 5′ end, and/or wherein the lengths of the first and second ends of the at least one DNA probe that are designed to hybridize to the RNA sample to the nucleotide stretches of known identity are in the interval of 10-30 nucleotides.

According to some embodiments, the means for sequencing the rolling circle amplification product comprises reagents, necessary probes and/or fluorophores for sequencing the amplified circularized oligonucleotide.

According to some embodiments, the means for library preparation comprises reagents for preparing a library of profiles of mutations, insertions, deletions and/or single or nucleotide variations in the RNA sample.

According to some embodiments, the means for probe extraction from RNA sample comprises reagents for extracting the ligated probes from RNA sample, purification, downstream PCR amplification and sequencing via NGS.

Hereby, profiling data can be gathered and structured in order to provide a library of profiled identities of RNA samples corresponding to a mutation, insertion, deletion or other variation of the RNA samples.

Effects and features of the second aspect are to a large extent analogous to those described above in connection with the first aspect. Embodiments mentioned in relation to the first aspect are largely compatible with the second aspect.

To summarize some of the key features and advantages of the present disclosure:

The present disclosure provides:
A method for Gap filling of at least one DNA probe, such as a padlock probe using mRNA directly as a template.
The selection of enzymes to perform the gap-filling reaction (polymerases with Reverse Transcriptase activity, and limited strand displacement). The current preferred choices are Bst DNA polymerase Full Length, as well as DNA Pol 1, TTH DNA Pol and engineered mutant polymerases derived from the thermophilic bacterium Bacillus stearothermophilus family of polymerases and other polymerases with RT activity which will be further tested.
Selection of the appropriate enzyme to perform the enzymatic nick sealing of the gap-filled DNA probe(s): a DNA ligase that can act efficiently using RNA as a splint molecule. The current choice is SplintR ligase, currently the only available commercial enzyme that is able to efficiently ligate DNA on an RNA splint.
Selection of the appropriate chemical reaction to perform the chemical nick sealing of the gap-filled DNA probe(s): click chemistry. The current choice is copper-catalyzed azide-alkyne cycloaddition (CuAAC) reaction that involves the use of a copper catalyst to join azide and alkyne functional groups for bioconjugation.
Combination of in situ RT and enzymatic ligation in the same step for increased efficiency (preferred for optimal efficiency).
The method can be used to capture and sequence unknown sequences flanked by known sequences on RNA also when solution mixtures (not in-situ). Potentially useful for in vitro diagnostics application.
Instead of merely being able to profile SNV/point mutations on mRNA in situ, one is also able to sequence in situ more complex variations (insertions/deletions, multiple substitutions) on a transcript of interest.

The present disclosure will become apparent from the detailed description given below. The detailed description and specific examples disclose preferred embodiments of the disclosure by way of illustration only. Those skilled in the art understand from guidance in the detailed description that changes and modifications may be made within the scope of the disclosure.

Hence, it is to be understood that the herein disclosed disclosure is not limited to the particular component parts of the device described or steps of the methods described since such device and method may vary. It is also to be understood that the terminology used herein is for purpose of describing particular embodiments only, and is not intended to be limiting. It should be noted that, as used in the specification and the appended claim, the articles “a”, “an”, “the”, and “said” are intended to mean that there are one or more of the elements unless the context explicitly dictates otherwise. Thus, for example, reference to “a unit” or “the unit” may include several devices, and the like. Furthermore, the words “comprising”, “including”, “containing” and similar wordings does not exclude other elements or steps.

### Definitions

The term “in-situ profiling” means measuring the abundance of variants (such as SNVs) in a biological sample that preserves the spatial information of the location of the variants.

The term “at least one DNA probe” is to be interpreted as one or more probes that can hybridize to an RNA sample and upon reverse transcription and ligation be amplified by rolling circle amplification. Examples include DNA padlock probes (as disclosed in detail in this disclosure) and linear probes (also referred to as “linearly sequenced probes”) that can be circularized by introducing a bridging probe and a ligase. Throughout the description, reference is typically made to padlock probes. However, at least two linear probes that are circularized by introducing a bridging probe and a ligase are also conceivable and is applicable for the purposes of the present invention. The introduction of a bridging probe and a ligase for ligating the at least two linear probes would typically take place before rolling circle amplification.

The term “sample” is to be interpreted as any biological tissue sample from any species, and/or cultured cells on e.g., microscope slides or coverslips.

The term “PLP” is an abbreviation of “padlock probe”.

The term “RCA” is an abbreviation of “rolling circle amplification”.

The terms “SNP” and “SNV” are abbreviations of “single nucleotide polymorphism” and “single nucleotide variation”, respectively. These terms both refer to variations/mutations of a single nucleotide position compared to a reference sequence (e.g., a wildtype), and in the context of this disclosure the terms “SNP” and “SNV” are used interchangeably.

The term “strand displacement” refers to the ability of a polymerase to displace downstream DNA/RNA encountered during synthesis. For the purposes of the present disclosure, related to gap filling applications, a low or limited strand displacement activity is preferred.

The term “nick translation” refers to the phosphorylation of a dephosphorylated 5′ DNA probe end (such as a 5′ padlock probe end), typically performed by an enzyme (a reverse transcriptase or polymerase) having a nick translation activity.

The term “CuAAC” refers to Copper-Catalyzed Azide-Alkyne Cycloaddition. CuAAC is a highly selective click chemistry reaction that involves the use of a copper catalyst to facilitate the reaction between azide and alkyne functional groups, resulting in the formation of a triazole link.

The term “NGS” refers to Next-Generation Sequencing, also known as high-throughput sequencing, is a powerful technology used to determine the nucleotide sequence of DNA in a highly efficient and cost-effective manner.

The term “downstream PCR amplification” refers to PCR amplification of the extracted ligated probe(s) from RNA sample after purification/enrichment, prior to NGS sequencing.

The term “LNA” nucleotides refers to a locked nucleic acid (LNA), also known as bridged nucleic acid (BNA), and often referred to as inaccessible RNA, which is a modified RNA nucleotide in which the ribose moiety is modified with an extra bridge connecting the 2′ oxygen and 4′ carbon.

### BRIEF DESCRIPTIONS OF THE DRAWINGS

The above objects, as well as additional objects, features and advantages of the present disclosure, will be more fully appreciated by reference to the following illustrative and non-limiting detailed description of example embodiments of the present disclosure, when taken in conjunction with the accompanying drawings.

FIG. 1 shows a negative control across 10 and 20 nt gaps—Phosphorylated Gap filling probe with ligase & no reverse transcriptase. RNA sample: 18srRNA.

FIG. 2 shows gap filling across 10 nt gap with ligase & different reverse transcriptases: (a) DNA Pol 1, (b) TTH DNA Pol, (c) BST. RNA sample: 18srRNA.

FIG. 3 shows gap filling across a 20 nt gap with ligase & BST as reverse transcriptase. RNA sample: 18srRNA.

FIG. 4 shows gap filling of a hybridised padlock probe with a reverse transcriptase.

FIG. 5 shows ligation of the hybridised and reverse transcribed padlock probe to create a circularized probe.

FIG. 6 shows rolling circle amplification of a circularized probe.

FIG. 7 shows an overall scheme of the method of the present disclosure.

FIG. 8 shows an overall scheme according to one embodiment of the method of the present disclosure, related to in situ probe activation/nick translation.

FIG. 9 shows an overall scheme of the method of the present disclosure using non-phosphorylated probes and nick translation.

FIG. 10 shows a gap filling probe design in accordance with the present disclosure.

FIG. 11 shows targeting of 18s rRNA with a 10 nt gapped PLP and ligation of phosphorylated PLP across a 10 nt gap, followed by RCA.

FIG. 12 shows targeting of 18s rRNA with PLP with 0 nt gap and ligation of non-phosphorylated PLP across a 0 nt gap, followed by RCA.

FIG. 13 shows targeting of 18s rRNA with PLP with 0 nt gap, and nick translation, followed by ligation of non-phosphorylated PLP across a 0 nt gap, followed by RCA.

FIG. 14 shows targeting of 18s rRNA with PLP with 10 nt gap, and primer extension, followed by nick translation and ligation of non-phosphorylated PLP across a 10 nt gap, followed by RCA.

FIG. 15 shows in situ single base extension sequencing of gap filled rolling circle product for gap filled identities.

FIG. 16 (a and b) shows in situ single base extension sequencing of rolling circle products of 18srRNA PLP (10 nt gap).

FIG. 17 shows probe design for exonuclease-resistant padlock probes and linear split probes for controlled gap filling on RNA with enzymatic ligation.

FIG. 18 shows probe design for padlock probes and linear split probes for gap filling on RNA with chemical click chemistry ligation.

FIG. 19 shows the general overview of the method and probe design for the profiling of mutation on RNA via gap filling.

### DETAILED DESCRIPTION

The present disclosure will now be described with reference to the accompanying drawings, in which preferred example embodiments of the disclosure are shown. The disclosure may, however, be embodied in other forms and should not be construed as limited to the herein disclosed embodiments. The disclosed embodiments are provided to fully convey the scope of the disclosure to the skilled person.

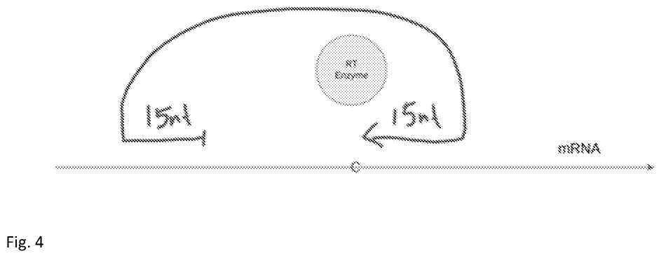

The present disclosure presents a method and a kit for use in the method to profile mutations and insertions or deletions directly on mRNA on biological samples while preserving tissue morphology. The invention will now be described with reference to FIGS. 4-7 and FIG. 19.

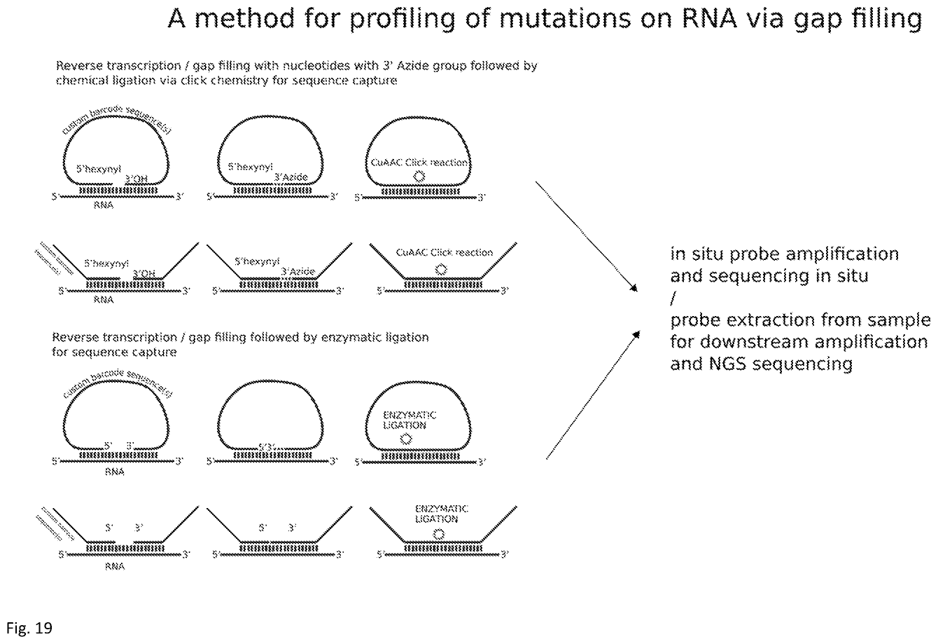

To start, an RNA sample is provided, which RNA sample comprises at least one mutation, insertion, deletion or variation in the form of a stretch of unknown identity to be profiled. DNA Probe(s) can be designed to hybridize to the sequences flanking the stretch of unknown identity, before a gap filling/reverse transcription reaction and ligation reaction is performed. The resulting probe encapsulating the now reverse transcribed nucleotides will be encapsulated in the probe(s) and the identities of the unknown stretch can be interrogated in situ or be extracted from the RNA sample, downstream PCR amplified and sequenced via NGS. (see FIG. 19)

Where an RNA sample is provided, which RNA sample comprises at least one mutation, insertion, deletion or variation in the form of a stretch of unknown identity to be profiled. In this example, the RNA sample is mRNA, and the stretch of unknown identity is upstream (to the left) of a known position (illustrated with a “C”) (see FIG. 4).

Depending on the length of the stretch of unknown identity, at least one DNA probe, e.g., a padlock probe is designed which upon hybridization to the RNA sample exhibits a gap between the 3′ and the 5′ end. In this example, the length of the 3′ and 5′ arms that hybridize to the RNA sample are 15 nucleotides.

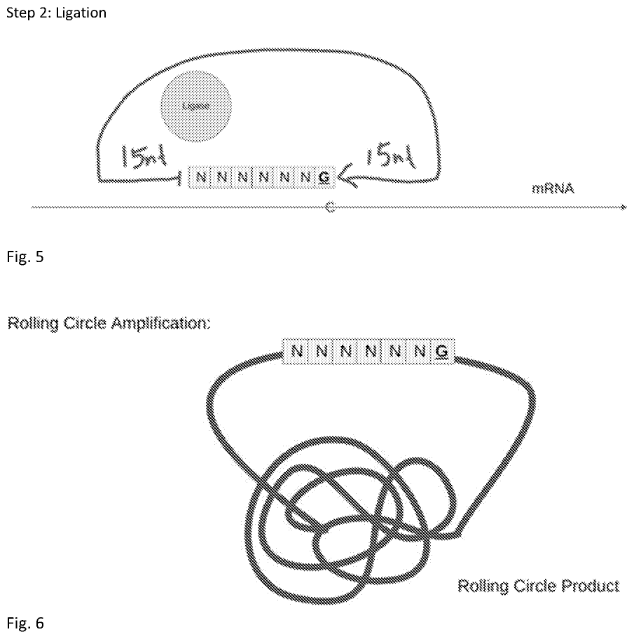

The hybridized padlock probe is gap filled using a reverse transcriptase (RT enzyme) with low strand displacement activity and/or good RT fidelity (see FIG. 4-5). The 3′ end of the padlock probe hybridizes to the last known base and the RT interrogates the unknown sequence(s) to be encoded into the padlock probe. Thus, nucleotides of (yet) unknown identity, corresponding to the unknown positions of the RNA sample, are added to the growing 3′ end, until the growing 3′ end reaches the 5′ end, and nucleotides corresponding to the entire stretch of unknown identity in the RNA sample have been added. In this example, this is represented by the sequence “NNNNNNG”, wherein “N” denotes a nucleotide with (yet) unknown identity.

In the next step (see FIG. 5), the nick between the 5′ end and the 3′ end of the hybridized padlock probe is sealed by adding a ligase. The ligase seals the 3′end with the 5′ end so that a circularized padlock oligonucleotide comprising a sequence complementary to the stretch of unknown identity of the RNA sample is obtained.

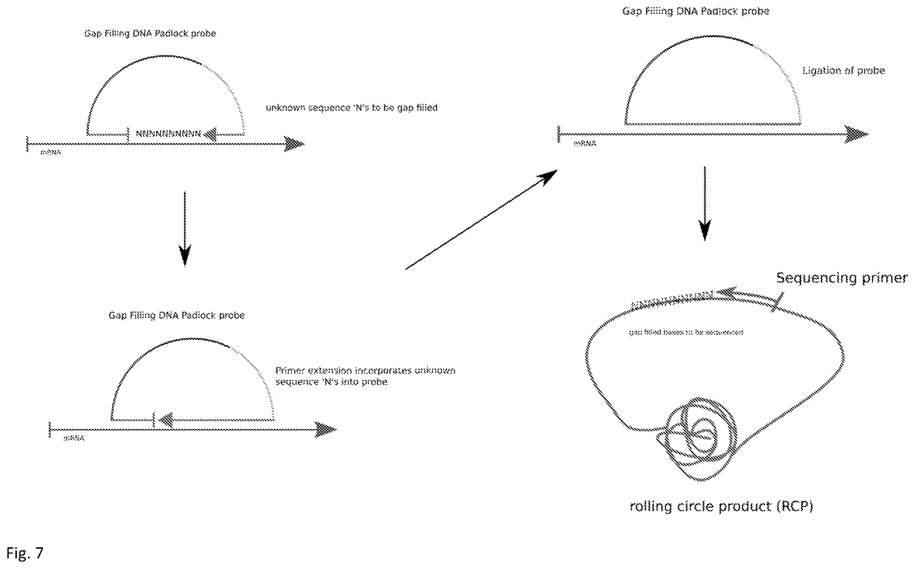

The circularized padlock oligonucleotide is thereafter amplified (see FIG. 6) by rolling circle amplification, thereby obtaining multiple copies of the circularized padlock oligonucleotide (rolling circle product (RCP)).

The rolling circle product can finally be in-situ sequenced using e.g. either sequencing by synthesis (https://www.ncbi.nlm.nih.gov/pmc/articles/PMC5829746/) or sequencing by ligation (https://academic.oup.com/nar/article/43/22/e151/1805334?login=true), to reveal the full sequence of the closed gap (in this case the “NNNNNNG” sequence), and thereby the identity of the unknown stretch in the RNA sample, i.e. the mutation, insertion, deletion and/or variation in the RNA sample, which is complementary to the sequence of the closed gap.

FIGS. 7 and 19 shows an overall scheme of the method of the present disclosure.

### The RNA Sample

In the present disclosure, the at least one stretch of unknown identity of the RNA sample comprises at least one nucleotide position to be profiled wherein the at least one nucleotide position to be profiled is an insertion, deletion and/or a single or multiple nucleotide variation in the RNA sample. The RNA sample can be an mRNA, rRNA or other non-coding RNA sample.

The RNA sample can be obtained from any tissue or cell line of choice, and e.g., be pretreated as exemplified in Example 4. The skilled person would be aware of alternative ways of extracting and/or pretreating the sample material.

For example, tumor sections are of interest to profile for mutations in the tumor micro environment for immune-oncology applications. However, a plurality of other applications can also be contemplated.

In order for the RNA sample to be used in the method of the present disclosure, at least parts of the RNA sample must include a known sequence of nucleotides, so that at least one DNA probe (such as a padlock probe, as explained below) can be designed to hybridize to the RNA sample. At least, stretches of nucleotide of the RNA sample corresponding to the length of the parts of the at least one DNA probes that are designed to hybridize to the RNA sample (i.e., the 3′ and 5′ arms) must be known. Also, in some embodiments the RNA sample comprises at least one nucleotide position to be profiled, i.e., a position wherein a mutation, insertion deletion and/or a single nucleotide variation (or polymorphism) (SNV/SNP) of interest is to be identified. Thus, the mutation must be flanked at both sides by known sequences, so that at least one DNA probe can be designed to hybridize both 3′ and 5′ to the SNV. Alternatively, in some embodiments the RNA sample comprises a 0 nt gap to be profiled, i.e., one identifies exactly what one knows is there, which is beneficial when coupled with the use on non-phosphorylated probes, where the probes are only activated when there is a 100% match. This greatly reduces off target detection.

### The Padlock Probe and Related Technology

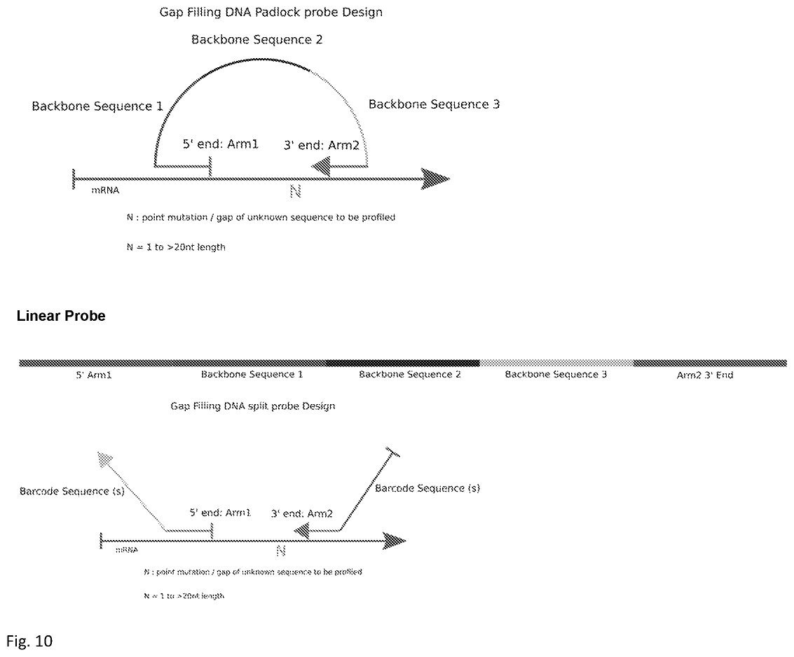

The general design of padlock probes is as shown in FIG. 10. The technology of designing chimeric padlock probes in general is known in the art. However, for this disclosure the following design of gap filling PLP is used:
1. First, a gap filling PLP design encompasses 2 arms (Arm1 & Arm2) that are complementary to a stretch of known mRNA sequence, flanking a gap of unknown sequence to be profiled ‘N’. The total combined length of Arm1 & Arm2 should be 30-50 nt in length (15-25 nt per arm for a symmetric design). It is also possible for one to design asymmetrical gap filling PLPs depending on one's application.
2. The 3′ end of the PLP should sit right before the stretch of unknown sequence ‘N’ 3. The unknown sequence ‘N’ can e.g., be from 1->20 nt in length, depending on one's application.
4. Two (2) or more unique backbone sequences can be introduced by the user which includes an RCA priming site to allow for an RCA primer to anneal and potentially any other unique sequences to facilitate probe identification downstream after amplification.

In the present disclosure, the first end of the padlock probe is designed to hybridize to the RNA sample to the nucleotide stretch of known identity at the 3′ side of the nucleotide stretch of unknown identity, and the second end of the padlock probe is designed to hybridize to the RNA sample to the nucleotide stretch of known identity at the 5′ side of the nucleotide stretch of unknown identity. The padlock probe can be either phosphorylated or non-phosphorylated at the 5′ end. Using a padlock probe that is non-phosphorylated at the 5′ end will require using an enzyme having a nick translation activity in order to seal the padlock probe. In embodiments, a reverse transcriptase having the nick translation activity can be used.

In some embodiments, the 3′ end and/or the 5′ end of the probe is hybridized to the RNA sample by at least 6, 7, 8, 9, 10, 11, 12, 13, 14 or at least 15 nucleotides. Thus, even though perfect hybridization (in terms of number of nucleotides of the probe(s) hybridizing to the RNA sample) may not be achieved, the subsequent reactions may still be performed to a sufficient degree.

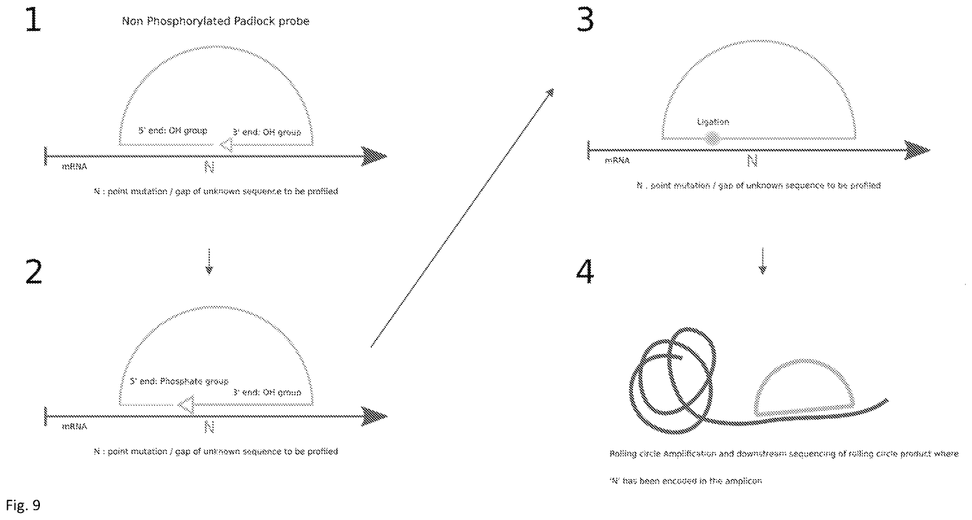

FIG. 9 describes the use of non-phosphorylated probes. In step 1, hybridization of the non-phosphorylated PLP to the RNA sequence of interest (mRNA in this case) is performed. In step 2, a reverse transcriptase/polymerase having a RT activity is used to extend the 3′ end of the PLP, thereby encoding (yet unknown) nucleotides (“N”) into the probe while the nick translation activity of the reverse transcriptase phosphorylates the 5′ end of the probe. In step 3, the PLP is circularized using a ligase. In step 4, the circularized PLP is amplified by RCA and the identity of the rolling circle product can be sequenced.

Using non-phosphorylated padlock probes significantly decreases the off-target detection due to high ligase activity (such as by using SplintR). This is especially significant when using a probe to gap-fill a gap of 1 nucleotide, where SplintR is able to ligate even if the RT enzyme (such as BST) does not perform primer extension, which encodes the point mutation into the PLP.

Typically, the lengths of the first and second ends of the padlock probe that are designed to hybridize to the RNA sample to the nucleotide stretches of known identity are in the interval of 10-30 nucleotides. However, the length of the first and second ends of the padlock probe can be shorter and longer, as long as a proper hybridization to the RNA sample is obtained.

Also, the gap between the first and second ends of the DNA padlock probe hybridized to the RNA sample, corresponding in length to the stretch of unknown identity to be identified, is typically in the interval of 1-50, 1-40, 1-30 or 1-20 nucleotides, as long as a proper reverse transcription and sealing of the gap in the padlock probe is obtained.

For this context, the choice and design of padlock probes need to take account for the mutation or variation to be profiled. Some different situations may occur which can vary the choice of padlock probes to design and use:

In a first situation, the RNA sample comprises one mutation or variation to be profiled, i.e., one stretch of unknown identity to be profiled. The identity of this stretch that is typically 1-20 nucleotides long is unknown and each position can vary between all four bases (A, G, C, U).

Typically, one padlock probe is designed and hybridized to the RNA sample, and subsequently the hybridized padlock probe is ligated to become circularized, which circularized padlock probe is amplified and sequenced to reveal the identity of the unknown stretch of nucleotides.

In a second situation, the RNA sample comprises a plurality of mutations and/or variations to be profiled, i.e., a plurality of stretches of unknown identity to be profiled. The identity of each stretch that is typically 1-20 nucleotides long is unknown and each position in each stretch can vary between all four bases (A, G, C, U). Typically, one padlock probe is designed for each stretch of unknown identity to be profiled and hybridized to the RNA sample, and subsequently the hybridized padlock probes are ligated to become circularized, which circularized padlock probes are amplified and sequenced to reveal the identity of the unknown stretches of nucleotides.

For typical conditions allowing hybridization of padlock probes to the RNA sample, details are provided below in the example section.

In some embodiments, an anchoring sequence is also included in the padlock oligonucleotide, for accessing amplification events, for a quick quality control.

### Linear Probes

As an alternative to using padlock probes and the related technology, linear probes (also referred to as “linearly sequenced probes” or “split probes”, illustrated in FIG. 10) can be used. In such case, typically two linear probes are introduced, which probes each hybridize to the sequences of known identity flanking the at least one nucleotide stretch of unknown identity, thereby resulting in a gap between the ends of the hybridized first and second probes. Thus, one of the two linear probes will hybridize 3′ of the gap, and one will hybridize 5′ of the gap. Hence, the 5′ end of the linear probe hybridizing 3′ of the gap corresponds to the 5′end of a padlock probe, and the 3′end of the linear probe hybridizing 5′ of the gap corresponds to the 3′ end of a padlock probe, for the purposes of this disclosure. Hybridization, reverse transcription, nick translation and ligation will occur similarly as for padlock probes. Thereafter, a bridging probe and a ligase, and possibly other ingredients, can be used to circularize the linear probes and prepare for rolling circle amplification. Also, linear probes post ligation can be used in an in vitro capture application such as single cell sequencing.

### Reverse Transcription

The reverse transcriptase or polymerase with reverse transcriptase activity will typically have one or more of the following characteristics:
either being an RNA/a DNA polymerase or reverse transcriptase
the ability to synthesize a complementary strand in the presence of a primer (DNA or RNA) with RNA as a template
low error rate
proofreading activity
being able to perform primer extension from a nick
nick translation activity

For example, the polymerase with reverse transcriptase activity can be chosen from the group comprising BST DNA polymerase (Full Length), TTH DNA Polymerase and DNA Polymerase I, polymerases exhibiting low strand displacement activity and/or good RT fidelity as well as from other engineered mutant polymerases/reverse transcriptase from the Bacillus stearothermophilus family/polymerases derived from the thermophilic bacterium Bacillus stearothermophilus/Moloney Murine Leukemia Virus (M-MuLV, MMLV). The level of RT (reverse transcriptase) fidelity is related to the error rate of the enzyme and can e.g., be understood from the manufacturer or the manufacturer's instructions of the specific RT.

### Enzymatic Ligation, Click Chemical Ligation, Nick Sealing and Nick Translation

### Enzymatic Ligation

The ligase to be used will typically have one or more of the following characteristics:
the ability to efficiently catalyzing the formation/ligation of a phosphodiester bond between adjacent nucleotides with 5′ phosphate and 3′ hydroxyl groups
the ability to efficiently catalyzing the ligation activity of DNA/RNA probes splinted by complementary DNA/RNA strands
the ability to catalyze a ligation reaction in 30 minutes or less.

The DNA ligase can be chosen from Splint R ligase (sold by NEB), PBCV-1 DNA ligase and/or Chlorella virus DNA ligase, as well as from other ligases exhibiting suitable properties, such as high or very high ligation activity, e.g., as per described by the manufacturer. Typically, an affinity for RNA-splinted DNA substrates based on apparent Km being in the order of 1 nM is preferred.

The steps of reverse transcribing and sealing the hybridized DNA probe (such as a padlock probe) can be combined, whereby necessary reagents including required enzymes (reverse transcriptase and ligase) are added simultaneously, or at least shortly after each other. Combining these steps would facilitate making the method even more efficient.

Moreover, an additional step of nick translation can be performed after the in situ reverse transcription and before the nick sealing (ligation). In embodiments, nick translation is combined with the in situ reverse transcription step, or the ligation step, or both.

### Click Chemical Ligation

The click chemical ligation reaction used will typically be CuAAC reaction with the following characteristics:
The chemical ligation reaction is bio-orthogonal, meaning that it will proceed efficiently in the presence of biological molecules such as proteins, nucleic acids and other cellular components without affecting them.
The ability to efficiently catalyze the formation of covalent a triazole bond/link between adjacent nucleotides with 5′ alkyne and 3′ azide groups.

The steps of reverse transcribing the gap of unknown sequence(s) will be performed with nucleotides with 3′ azide group, such that post reverse transcribing, the 3′ end of the probe(s) will contain a 3′ azide group, which can then be chemically ligated to the adjacent 5′ alkyne group. Also, there are various types of click chemistries, other than the copper azide alkyne click chemistry discussed above. Other types of click chemistries such as Staudinger ligation, Strain-Promoted Azide-Alkyne Cycloaddition (SPAAC), Thiol-Ene Reaction, Thiol-Yne Reaction, Diels-Alder Reaction, Inverse-Electron-Demand Diels-Alder (IEDDA) can also be used in the context of this disclosure.

### Nick Translation

Nick translation is used for “activation” of DNA probes (such as padlock probes), in order to increase specificity under promiscuous ligation conditions.

Direct profiling of mRNA using padlock probes has a general problem of specificity: most enzymes ligating DNA or RNA padlocks using RNA as a splint show high mismatch-tolerance.

Gap-filling, as described in the context of this disclosure, allows one to perform actual sequencing of unknown stretches between known sequences, thereby facilitating a general solution to the problem of mutation detection, covering both single or multi-nucleotide events. A potential challenge with gap-filling is that one needs to ligate two (2) DNA strands (the extended padlock probe's arms or 2 linear, split probes) onto an RNA template, necessitating the use of a ligase that efficiently ligates DNA ends splinted by RNA. If this ligase has a limited specificity, noise coming from un-extended padlocks may be challenging to distinguish from the true signal. This is because this ligase finds a phosphorylated 5′end in proximity of a free 3′ end, and even if they are not exactly adjacent, will find a way to seal that nick or gap anyway in some occasions.

By using the step of nick translation this can be improved.

A feature of the BST enzyme that is used by the present inventors for RT-gap filling, is that it is able to perform nick translation. When the 3′ end extends and meet the 5′ end of the padlock, it chews a nucleotide out if it, and fills it by extending the 3′ growing strand with 1 nt. This means the nick is progressively pushed towards the 5′ side of the probe.

Instead of hybridizing a phosphorylated padlock, the inventors use instead a non-phosphorylated one. However close, a non-phosphorylated 5′ end cannot ligate in any way to a 3′ end. This way the unspecific activity of the ligase is prevented.

When one adds BST, two things happen. First, any gap (if existing) between the padlock arms gets filled according to the BST fidelity. Second, once BST brings the 3′ end in touch with the 5′ non-phosphorylated end, it cleaves one nucleotide off from the latter, exposing a 5′-phosphate from the following 5′-nucleotide.

Only at this point the ligase (such as SplintR ligase) can seal the nick. So, in this setting, ligation (not very specific) becomes conditional to the nick translation (which should be instead very specific). As a result, we see a great gain in specificity even using a ligase with limited specificity, because the probes need to be somehow “activated” by the nick translation event.

Hereby, by using the approach of nick translation (or “probe activation”) a ligase having limited specificity can be made specific, and this ligase can be exploited for the purpose of accurately characterizing SNP or other types of mutations on RNA with a good degree of accuracy. Also, this really solves the problems of characterizing point mutation via gap-filling (i.e.: where you would require a gap-filling of 1 nt) which would be particularly prone to false results due to the ability of a ligase with low specificity to ligate even across short gaps.

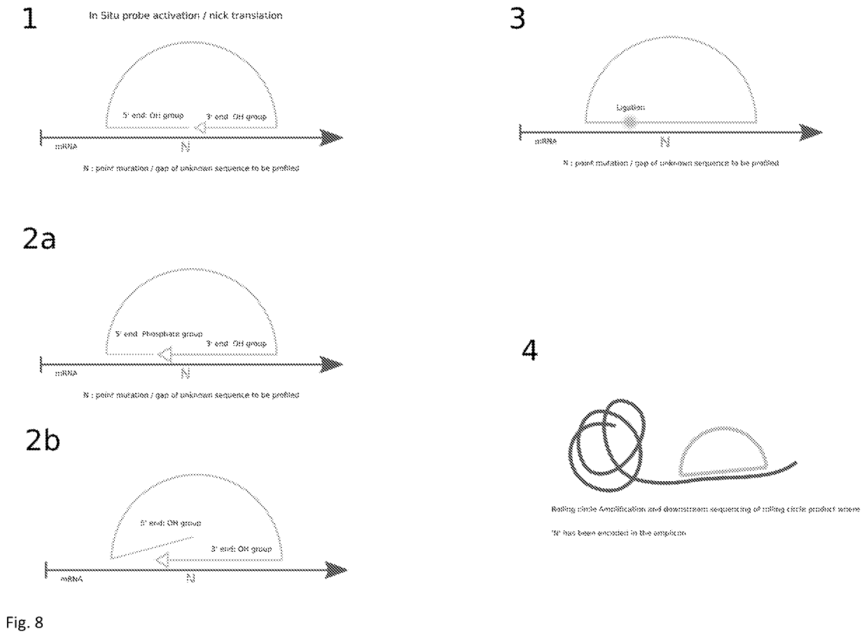

Referring to FIG. 8, the concept of in situ probe activation/nick translation is illustrated. (1) DNA probes with 5′ OH and 3′ OH groups are introduced to the target of interest. (2) (a) Should the DNA probe hybridize perfectly to its target, primer extension will occur from the 3′OH group and come in contact with the 5′ OH group, which will activate nick translation activity, phosphorylating the 5′ end of the probe, allowing ligation to happen, hence circularizing the probe (3) and allowing it to be amplified by rolling circle amplification or to be extracted from the RNA sample for downstream PCR amplification and sequenced via NGS. (2)(b) Should incomplete hybridization of the probe happen primer extension may occur from the 3′ OH group. Due to the incomplete hybridization, nick translation cannot happen, resulting in the partially hybridized probe to not be activated and hence unable to ligate and circularize, thus the probe will not be able to undergo rolling circle amplification.

In other embodiments, the approach of nick translation (or “probe activation”) can be extended to the use with linear, split probes.

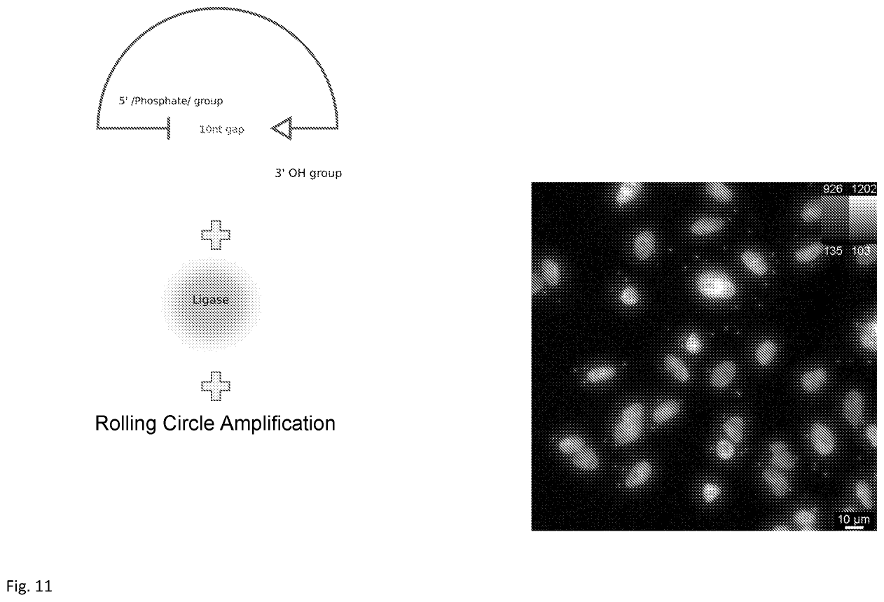

Examples 5-8 (FIGS. 11-14) illustrates these events.

### Controlled Gap Filling Using Nick Translation

According to some embodiments, the DNA probe(s) provided in step (b) comprises at least one phosphorothioate bond close to the 5′ end, and in step (c) (i) a polymerase with reverse transcriptase activity and nick translation activity, and (ii) a universal base primer, having a length corresponding to or slightly shorter than the gap, are added, wherein the universal base primer binds to the stretch of unknown identity of the RNA sample and the polymerase thereafter nicks and converts the bases of the universal base primer to bases complementary to the stretch of unknown identity of the RNA sample, after which the gap is sealed.

Hereby, the nick translation activity, when using a polymerase having such activity, can be controlled, and will avoid undesired displacement of the probe(s) thereby reducing capture efficiency.

In some embodiments, 1, 2, 3, 4, 5, 6, 7, 8, 9, 10, for example 3-7 and especially about 5 of the nucleotides at the 5′ end of the DNA probe comprise(s) phosphorothioate bonds. Typically, the phosphorothioate bonds are included at the 5′ end of the DNA probe starting from the second base from the 5′ end.

Hence, the introduction of phosphorothioate bonds near the 5′ end of the capture probes allows for one to control the nick translation activity of polymerases and that prevents the probe from being displaced.

Introduction of universal bases into the stretch of unknown sequence to be profiled allows for better sequence capture. The universal bases will then be converted to the ‘correct’ bases via nick translation instead of traditional reverse transcription.

The introduction of cleavable bases into the capture probe for linearization.

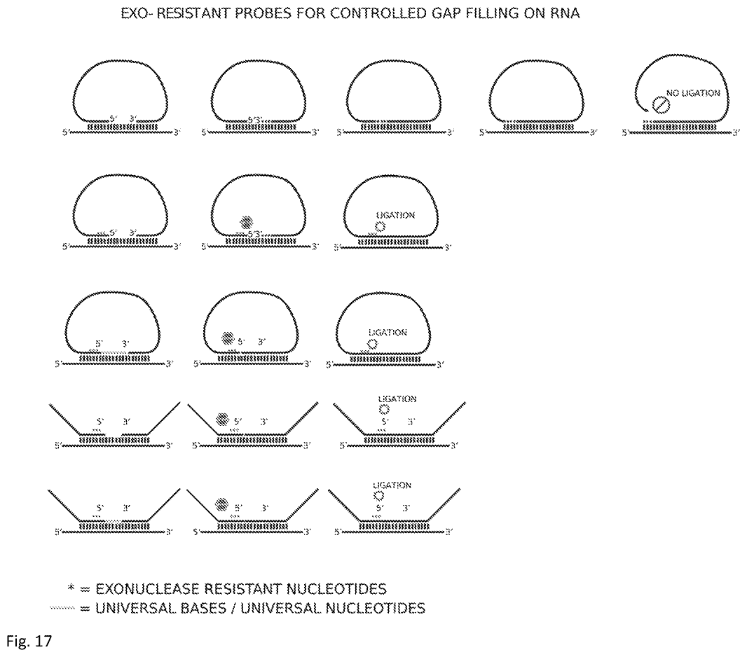

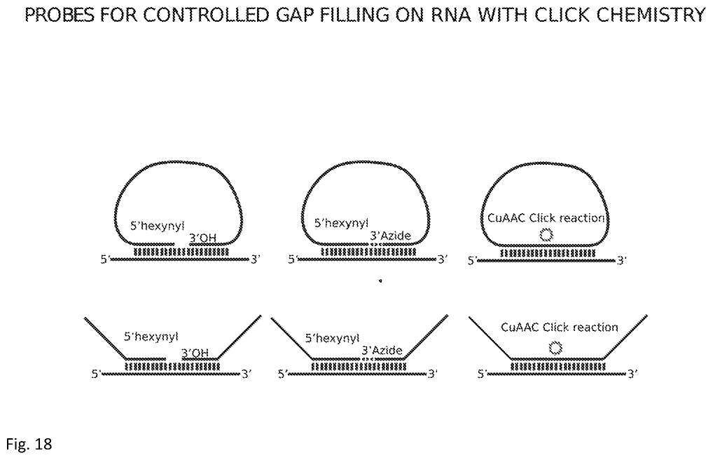

FIG. 17 shows the principles of this embodiment.
Top row: In some cases when using a polymerase with strand displacement activity, due to the use of polymerase for a gap filling approach using a circularizable probe/probe pair, it requires the probe(s) to hybridize to a known target sequence that flanks a region of unknown sequence to be profiled via gap filling. However, certain polymerases used for gap filling exhibit nick translation activity. This nick translation activity if uncontrolled, such as could occur when separating the gap filling and ligation steps into 2 separate steps, may result in the displacement of the of the probe(s) which reduces the capture efficiency.
Middle row: Phosphorothioate bonds have been introduced at the 5′ end of the probe. Hereby, strand displacement is prevented. However, the probe is not completely gap-filled and sealed.
Bottom row: Phosphorothioate bonds and universal bases/nucleotides have been introduced, resulting in no strand displacement, along with gap filling and sealing

The inventors have data showing that the incorporation of phosphorothioate (PS) bonds near the 5′ end of the padlock probe is able to allow control of the gap filling rate with polymerases that exhibit nick translation activity. The design and incorporation of PS bonds in the padlock probe prevents the 5′ end of the probe from being displaced with an uncontrolled RT and nick translation, preventing it from being circularized.

The probe can be a proprietary design that can be Incorporated into gap filling reagent kits that has applications for both in situ and in vitro applications.

### Rolling Circle Amplification

Circularized probes, such as circularized padlock probes, i.e., padlock probes that have undergone reverse transcription and ligation and linear probes that have been ligated with a splint post gap filling, and therefore includes a stretch of DNA bases that are complementary to each stretch of unknown identity in the RNA sample to be identified, will undergo amplification, typically using rolling circle amplification. For typical conditions allowing rolling circle amplification, details are provided below in the example section.

The (DNA) polymerase to be used to perform the rolling circle amplification reaction typically has on or more of the following characteristics:
high processivity, i.e., ability to generate large fragments
high fidelity, i.e., low error rate during replication
ability to tolerate RNA/DNA bases as templates
3-5′ proofreading activity
high strand displacement activity

In some embodiments, the primers, enzymes and other reagents for the steps of hybridization, reverse transcription, ligation and amplification (typically RCA) may be added in separate steps, or at least partly combined, thereby increasing efficiency of the method. For example, RCA primers may be added to the ligation mix to anneal to the hybridized PLPs.

In some embodiments, the enzyme/DNA polymerase selected for RCA is BST polymerase or Phi29 DNA polymerase and their derived engineered mutant form.

### Sequencing

The determination of the identity of the at least one stretch of unknown identity in the RNA sample, i.e., referring to the mutation, insertion, deletion and/or variation in the RNA sample is typically performed by using any suitable sequencing protocol. For example, sequencing for the determination of the identity of the at least one stretch of unknown identity can be performed in vitro, via NGS sequencing or Sanger sequencing of the extracted probes from the RNA sample. Sequencing can also be performed in situ, such as by Sequencing by Synthesis SBS, Sequencing by Ligation SBL, SOLiD sequencing or single base extension sequencing. Typically, the sensitivity of the sequencing method needs to be able to resolve single nucleotide differences. Moreover, the sequencing method used can be of some barcoding nature.

### Applications

The profiling of mutations via gap filling on RNA method of the present disclosure can be used in a plurality of applications, such as any of the following examples:
mutation profiling in single cell sequencing experiments
profiling of insertion or deletions such as CRISPR validation in single cell sequencing experiments
in situ mutation profiling
in situ sequencing of insertion or deletions, such as in situ CRISPR validation
in situ lineage tracing/barcoding applications
in vitro mutation profiling for diagnostics applications
the method can be commercialised as a kit catering to researchers' needs (see second aspect of the invention

Thus, the method can be used for any of the following applications: mutation profiling, sequencing of insertion or deletions, CRISPR validation, tracing, ibarcoding applications, miRNA profiling in both in situ and in vitro applications, profiling of unknown sequences in RNA in solution and/or in vitro mutation profiling for diagnostics applications.

### Kit

The second aspect of this disclosure relates to a kit for use in a method for both in situ and in vitro profiling of mutations, insertions, deletions and/or single nucleotide variations in a RNA sample according to the first aspect, wherein the RNA sample comprises at least one nucleotide stretch of unknown identity, flanked by nucleotide stretches of known identity, the kit comprising:
(i) one or more DNA probes, chosen from (a) at least two linear probes or (b) one or more DNA padlock probes having a first and a second end, which first end is designed to hybridize to the RNA sample to the at least one nucleotide stretch of known identity at the 3′ side of the nucleotide stretch of unknown identity, and the second end is designed to hybridize to the RNA sample to the at least one nucleotide stretch of known identity at the 5′ side of the nucleotide stretch of unknown identity;
(ii) a polymerase with reverse transcriptase activity and optionally limited strand displacement, optionally including necessary reagents and buffers;
(iii) a DNA ligase that can use RNA as a splint molecule, optionally including necessary reagents and buffers;
(iv) optionally reagents used to extract ligated probes from RNA sample for downstream PCR amplification for NGS sequencing;
(v) optionally one or more amplification primers and a polymerase for rolling circle amplification, and necessary reagents and buffers;
(vi) optionally means for sequencing the rolling circle amplification product; (vii) optionally means for library preparation, and
(viii) instructions for use.

In some embodiments, the polymerase with reverse transcriptase activity is chosen from the group comprising BST DNA polymerase Full Length, TTH DNA Polymerase and DNA Polymerase I, as well as polymerases exhibiting low strand displacement activity and engineered mutant polymerases from the Bacillus stearothermophilus family/polymerases derived from the thermophilic bacterium Bacillus stearothermophilus/Moloney Murine Leukemia Virus (M-MuLV, MMLV).

In some embodiments, the DNA ligase is chosen from Splint R ligase, PBCV-1 DNA ligase and/or Chlorella virus DNA ligase.

In some embodiments, the ligation is performed chemically via CuAAC click chemistry.

In some embodiments, the one or more DNA probes are designed to exhibit a gap in the interval of 0-50, 0-40, 0-30 or 0-20 nucleotides between the first and second end upon hybridization to the RNA sample. Typically, the DNA probe is either phosphorylated or unphosphorylated at the 5′ end. The lengths of the first and second ends of the DNA probe that are designed to hybridize to the RNA sample to the nucleotide stretches of known identity are typically in the interval of 10-30 nucleotides.

In some embodiments, the means for sequencing the rolling circle amplification product comprises reagents, necessary probes and/or fluorophores for sequencing the amplified circularized oligonucleotide.

### Library Preparation

The kit can also include means for library preparation in order to prepare a library or database of profiles of mutations, insertions, deletions and/or single or nucleotide variations in RNA samples, as well as associated data and parameters. In some embodiments, the means for library preparation comprises reagents for preparing a library of profiles of mutations, insertions, deletions and/or single or nucleotide variations in the RNA sample.

### EXAMPLES

The inventors successfully tested the invention across a 10 and 20 nucleotides gap, using an RNA (18s rRNA) as a target. The invention principle works, as shown below.

### Example 1—Negative Control

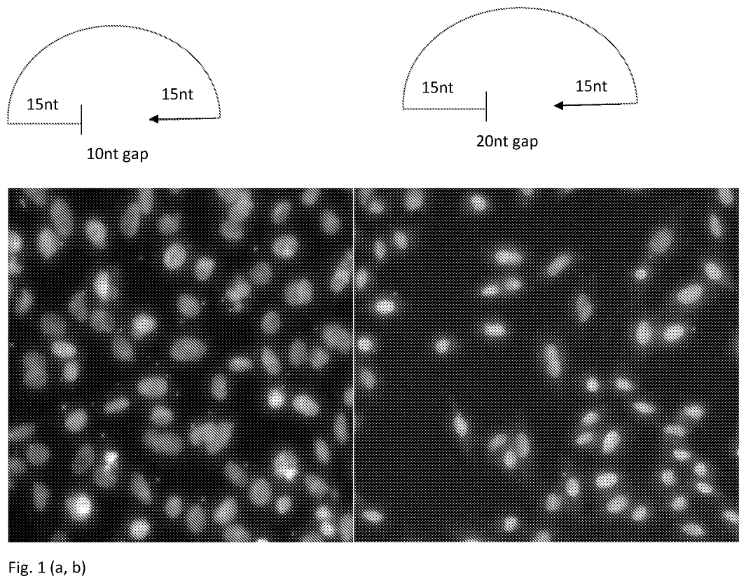

As a negative control (FIG. 1), the inventors omitted the RT enzyme in the reaction mix. Under these conditions no ligation of the padlock probe was expected, and complete absence of signal dots (in magenta). The inventors detect some negligible background signal, coming from a known promiscuous activity of SplintR ligase, which can ligate a minority of padlocks even across large gaps.

### Example 2—Gap Filling Across 10 nt Gap with Ligase & Reverse Transcriptase

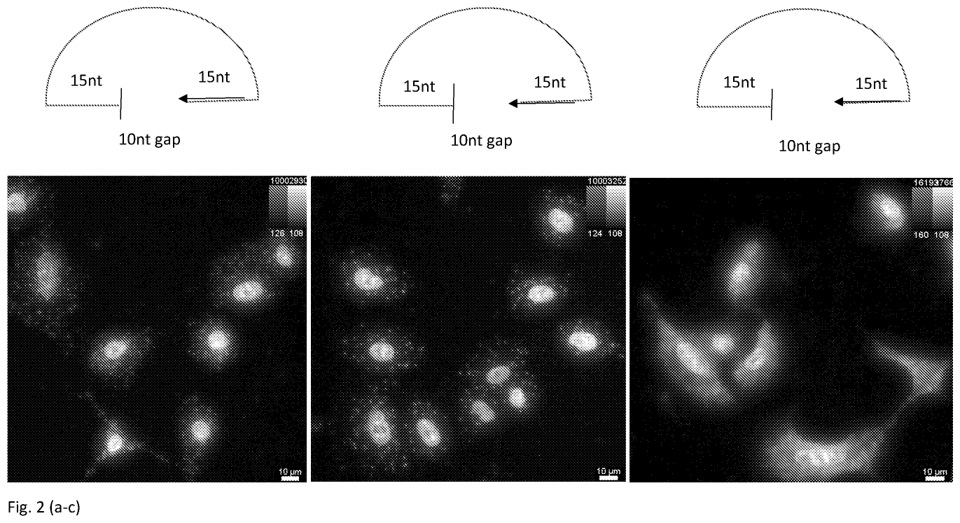

In this experiment the inventors incubate, together with the ligation, a set of reagents to perform the gap filling reaction (reverse transcriptase (DNA Pol 1 (a), TTH DNA Pol (b) and BST (c)), dNTPs and buffer). In these conditions, if the gap is filled, the inventors would expect a much higher signal concentration than in the negative control, which is indeed what was observed (FIG. 2). This is the proof of the principle of the idea underlying the present invention.

### Example 3—Gap Filling Across 20 nt Gap with Ligase & BST as Reverse Transcriptase

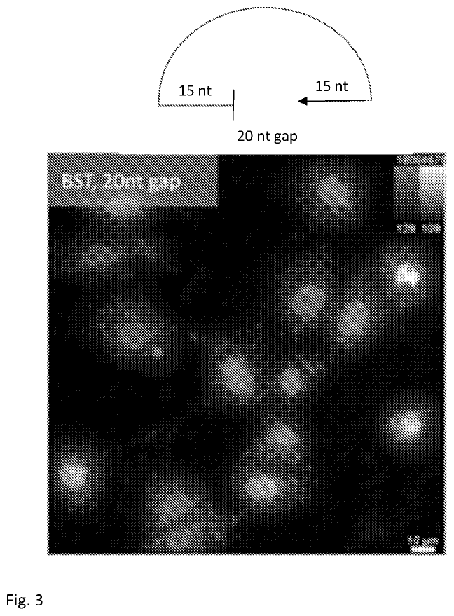

In this experiment the inventors incubate, together with the ligation, a set of reagents to perform the gap filling reaction (reverse transcriptase (BST), dNTPs and buffer). In these conditions, if the gap is filled, the inventors would expect a much higher signal concentration than in the negative control, which is indeed what was observed (FIG. 3). This is the proof of the principle of the idea underlying the present invention.

### Example 4—General Protocol for Gap Filling

The skilled person would understand that some reagents and conditions could be modified. The provided examples are thus for purposes of exemplifying the present invention.

A. Sample Pretreatment

Fresh frozen biological samples (can be cell line/any tissue that has been sectioned onto a microscope slide/coverslip) is first fixed with 3.7% formaldehyde or any other fixative e.g., Methanol, formalin etc.

If the sample is FFPE samples, dewaxing/de-crosslinking is first performed with Xylene and heat treatment (i.e., incubation at 45 degrees for 15 minutes).

The biological sample is then permeabilized with 0.1M HCl, with the addition of pepsin or proteinase K, or sodium dodecyl sulfate (SDS) or any other reagents typically used for permeabilization for in situ hybridization/immunohistochemistry experiments.

B. Hybridization

Probes (PLPs or pairs of linear probes) in a hybridization buffer is added to the RNA sample for probe hybridization.

TABLE 1
Hybridization Mixture

2X SSC
20% Formamide or Ethylene Carbonate
10 nM of each Probe

C. PLP hybridization is performed at 37-55° C. overnight. Hybridization may or may not be followed by washing steps to eliminate the excess unhybridized probes.

Reagents for reverse transcription, nick translation and ligation:

TABLE 2
Reagents

20 mM Tris-HCl
10 mM (NH₄)₂SO₄
10 mM KCl
0.05 mM ATP
2 mM MgSO₄
0.9 mM MnCl₂
0.1% Triton X-100
500 uM dNTPs
1 U/ul RiboProtect (RNase inhibitor)
0.1 U/ul BST Full Length
0.25 U/ul SplintR Ligase

Buffer pH~8.8 @25° C.

RT and ligation are performed at 37° C. for 2 h.

D. Rolling Circle Amplification (RCA)

RCA reaction buffer composition:

TABLE 3
RCA Buffer composition

50 mM Tris-HCl
10 mM MgCl₂
10 mM (NH₄)₂SO₄
5% Glycerol
0.25 mM dNTPs
0.2 μg/ml BSA
0.05 μM RCA primer
1 U/μl Phi29 Polymerase

RCA buffer pH~8.3 @25 C.

RCA is performed at 30 C overnight or 37 C for 5 h.

### Example 5—Targeting 18s rRNA, with a 10 nt Gapped Phosphorylated PLP

See FIG. 11, which discloses targeting of 18s rRNA with a 10 nt gapped padlock probe, having a phosphate group at the 5′ end. Ligation of the phosphorylated padlock probe is performed across the 10 nt gap, followed by rolling circle amplification. As can be seen, ligation is still possible across a 10 nt gap although not very efficiently in the absence of a primer extension reaction, resulting in some false detection events.

Thus, this example is to be viewed as a negative control experiment showing that there would be more unspecific signal of one were to use 5′ phosphorylated probes vs non-phosphorylated probes in the presence of SplintR, illustrating that probe activation via nick translation adds a layer of specificity to the data generated.

### Example 6—Targeting 18s rRNA, with a 0 nt Gapped Non-Phosphorylated PLP, Using a RT without Nick Translating Activity

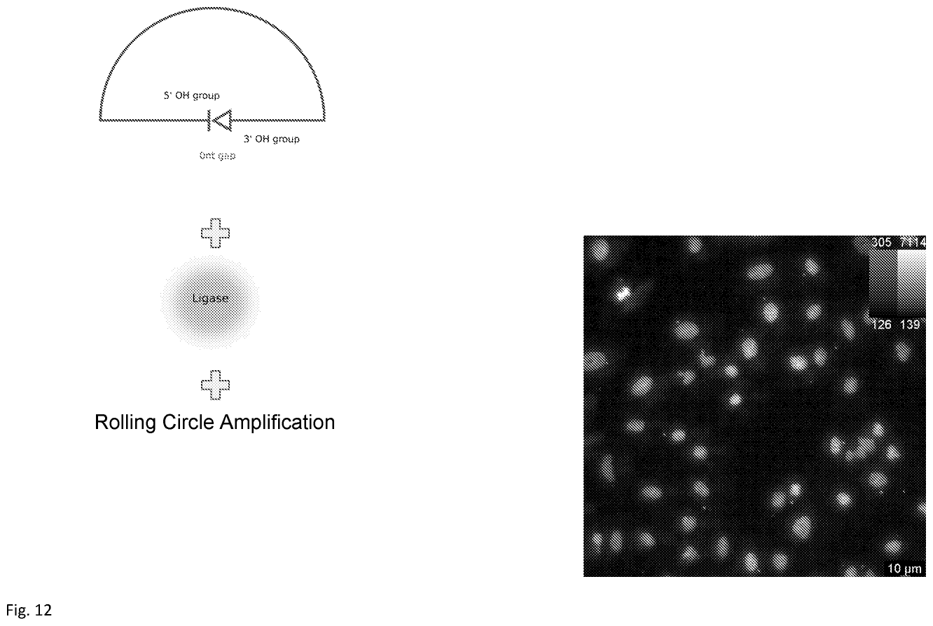

See FIG. 12, which discloses targeting of 18s rRNA with a 0 nt gapped padlock probe, being non-phosphorylated at the 5′ end. Ligation of the non-phosphorylated padlock probe is performed across the 0 nt gap, followed by rolling circle amplification. As can be seen, ligation is not successful, since the ligase is not able to ligate the non-phosphorylated 5′ end.

Thus, this example is a negative control experiment illustrating that using a non-phosphorylated PLP with 0 nt gap, ligation cannot occur despite using a very active and promiscuous SplintR ligase as the 5′ end of the padlock probe is not phosphorylated.

### Example 7—Targeting 18s rRNA, with a 0 nt Gapped Non-Phosphorylated PLP, Using a RT with Nick Translating Activity

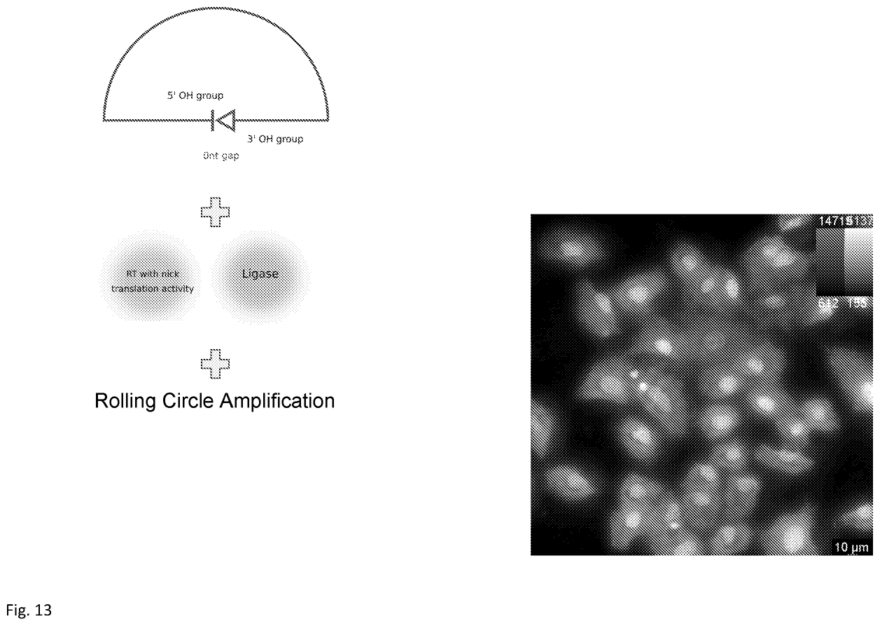

See FIG. 13, which discloses targeting of 18s rRNA with a 0 nt gapped padlock probe, being non-phosphorylated at the 5′ end. Nick translation (by a reverse transcriptase having nick translation activity), i.e., phosphorylation of the 5′ end (i.e., probe activation), is performed, followed by ligation of the padlock probe across the 0 nt gap, followed by rolling circle amplification. As can be seen, ligation is successful, as a result of using a reverse transcriptase with nick translation activity.

### Example 8—Targeting 18s rRNA, with a 10 nt Gapped Non-Phosphorylated PLP, Using a RT with Nick Translating Activity

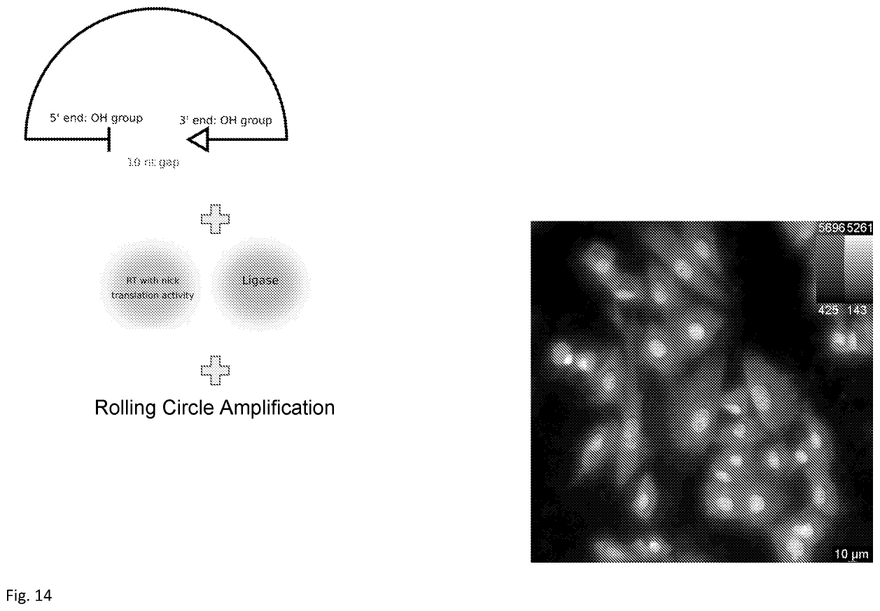

See FIG. 14, which discloses targeting of 18s rRNA with a 10 nt gapped padlock probe, being non-phosphorylated at the 5′ end. Primer extension followed by nick translation (by a reverse transcriptase having nick translation activity), i.e., phosphorylation of the 5′ end, is performed, followed by ligation of the extended and nick translated padlock probe, followed by rolling circle amplification. As can be seen, ligation is successful, as a result of using a reverse transcriptase for primer extension and nick translation.

### Example 9—In Situ Single Base Extension Sequencing of Gap Filled Rolling Circle Product

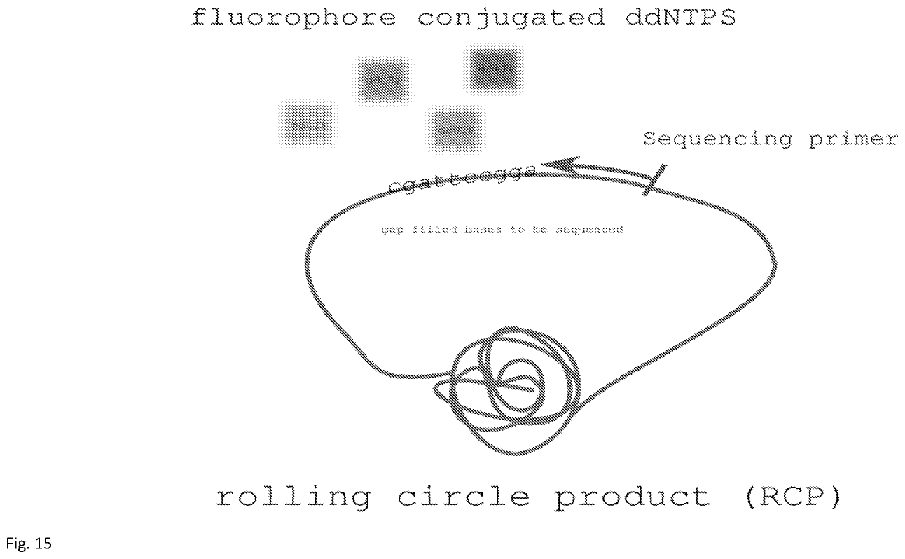

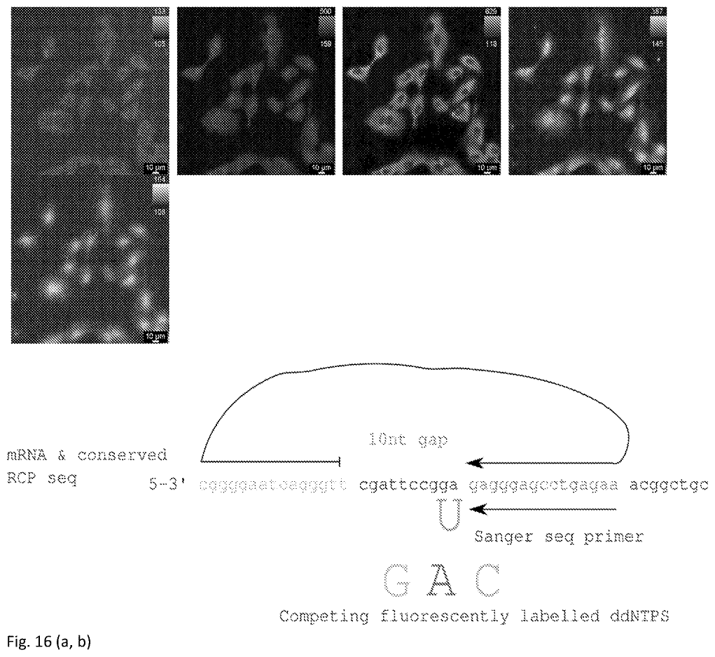

FIGS. 15 and 16 shows in situ single base extension sequencing of generated RCP for gap filled identities. In FIG. 15 it is shown that upon hybridization of a sequencing primer to the rolling circle product, fluorophore conjugated ddNTPs are added and detected (FIG. 16). See FIG. 16 for detection results, wherein a “U” was expected, should the gap filling reaction be performed correctly by the reverse transcriptase. The expected result was confirmed, since the base incorporated was ddUTP (corresponding to the green channel), with no off-target signal detected for the other fluorescent channels. “DAPI” (4′, 6-diamidino-2-phenylindole) stands for a DNA-specific probe, which forms a fluorescent complex by attaching to the DNA molecule.

The person skilled in the art realizes that the present disclosure is not limited to the preferred embodiments described above. The person skilled in the art further realizes that modifications and variations are possible within the scope of the appended claims. Additionally, variations to the disclosed embodiments can be understood and effected by the skilled person in practicing the claimed disclosure, from a study of the drawings, the disclosure, and the appended claims.

1. A method for profiling of mutations, insertions, deletions and/or single or nucleotide variations in an RNA sample, comprising the steps of:
(a) providing an RNA sample comprising at least one nucleotide stretch of unknown identity to be identified, flanked by nucleotide stretches of known identity;
(b) contacting the RNA sample with at least one DNA probe, such as at least two linear probes or at least one DNA padlock probe, having a first and a second end under conditions and with reagents allowing hybridization, which first and second ends are designed to hybridize to the sequences of known identity flanking the at least one nucleotide stretch of unknown identity, thereby resulting in a gap between the hybridized first and second ends of the at least one DNA probe;
(c) adding a polymerase with limited reverse transcriptase activity, and optionally nick translation activity, and optionally limited strand displacement, under conditions and with reagents allowing reverse transcription, thereby allowing reverse transcription of the at least one DNA probe from the first to the second end using the RNA sample as a template, so that the reverse transcribed part of the at least one DNA probe comprises the complementary sequence to the nucleotide stretch of unknown identity in the RNA sample;
(d) adding a ligase that can use RNA as a splint molecule under conditions and with reagents allowing ligation, or using chemical ligation such as click chemistry, thereby allowing sealing of the at least one reverse transcribed DNA probe in order to generate at least one linear or circularized oligonucleotide;
(e) amplifying the at least one linear or circularized oligonucleotide under conditions and with reagents allowing rolling circle amplification, thereby generating at least one amplified circularized oligonucleotide, or extracting the ligated probe from the RNA sample for downstream PCR amplification; and
(f) sequencing the at least one amplified oligonucleotide, thereby revealing the identity of the nucleotide stretch of unknown identity of the RNA sample.

2. The method according to claim 1, wherein the stretch of unknown identity of the RNA sample comprises at least one nucleotide position to be profiled wherein the at least one nucleotide position to be profiled is an insertion, deletion and/or a single or multiple nucleotide variation in the RNA sample.

3. The method according to any one of the preceding claims, wherein the RNA sample is an mRNA, rRNA or other non-coding RNA sample.

4. The method according to any one of the preceding claims, wherein the at least one DNA probe is chosen from (i) at least one padlock probe, (ii) at least two linear probes that can be circularized by introducing a bridging probe and a ligase, (iii) at least one padlock probe or at least two linear probes that can be enzymatically ligated and can be released from the RNA sample, amplified and sequenced via NGS (next generation sequencing), or (iv) at least one padlock probe or at least two linear probes that can be chemically ligated via click reaction, thereby not being circularized and can be released from the RNA sample, amplified and sequenced via NGS.

5. The method according to any one of the preceding claims, wherein the first end of the at least one DNA probe is designed to hybridize to the RNA sample to the nucleotide stretch of known identity at the 3′ side of the nucleotide stretch of unknown identity, and the second end of the at least one DNA probe is designed to hybridize to the RNA sample to the nucleotide stretch of known identity at the 5′ side of the nucleotide stretch of unknown identity.

6. The method according to any one of the preceding claims, wherein the at least one DNA probe is either phosphorylated or unphosphorylated at the 5′ end.

7. The method according to any one of the preceding claims, wherein the lengths of the first and second ends of the at least one DNA probe that are designed to hybridize to the RNA sample to the nucleotide stretches of known identity are in the interval of 10-30 nucleotides.

8. The method according to any one of the preceding claims, wherein the gap between the first and second ends of the at least one DNA probe hybridized to the RNA sample, corresponding in length to the stretch of unknown identity to be identified, is in the interval of 0-20 nucleotides.

9. The method according to any one of the preceding claims, wherein the polymerase with reverse transcriptase activity is chosen from the group comprising BST DNA polymerase (Full Length), TTH DNA Polymerase and DNA Polymerase I, as well as engineered mutant polymerases derived from the thermophilic bacterium Bacillus stearothermophilus, family A of DNA polymerases and other polymerases and reverse transcriptases exhibiting low strand displacement activity and/or good RT fidelity.

10. The method according to any one of the preceding claims, wherein the polymerase with reverse transcriptase activity is a DNA polymerase with limited reverse transcription activity and nick translation activity that is derived from the thermophilic bacterium Bacillus stearothermophilus or family A of DNA polymerases.

11. The method according to any one of the preceding claims, wherein the ligase is chosen from Splint R ligase, PBCV-1 DNA ligase and/or Chlorella virus DNA ligase.

12. The method according to any one of the preceding claims, wherein the hybridized 5′ end of the DNA probe(s) in step (b) contains a 5′-alkyne group and after reverse transcription with nucleotides with a 3′-azide group, the nick between adjacent 5′-alkyne and 3′-azide group is to be chemically ligated via CuAAC click chemistry.

13. The method according to any one of the preceding claims, wherein steps (c) and (d) are combined, thereby allowing reverse transcription and sealing of the at least one DNA probe within the same step.

14. The method according to any one of the preceding claims, wherein an additional step of nick translation is performed together with the reverse transcription of step (c) and before the nick sealing of step (d).

15. The method according to any one of the preceding claims, wherein the DNA probe(s) provided in step (b) are non-phosphorylated, and the combination of steps (c) and (d) where the reverse transcription and nick translation is conditional for probe activation, before nick sealing can occur.

16. The method according to claim 14 or 15, wherein the DNA probe(s) provided in step (b) comprises at least one exo-nuclease resistant nucleotide close to the 5′-end, and wherein in step (c) (i) a polymerase with reverse transcriptase activity and nick translation activity, and (ii) a universal base primer, having a length corresponding to or being slightly shorter than the gap, are added, wherein the universal base primer binds to the stretch of unknown identity of the RNA sample and the polymerase thereafter nicks and converts the bases of the universal base primer to bases complementary to the stretch of unknown identity of the RNA sample, after which the gap is sealed.

17. The method according to claim 16, wherein 1-10, 3-7 or about 5 consecutive nucleotides at the 5′ end of the DNA probe comprise(s) phosphorothioate bonds.

18. The method according to claim 17, wherein at least 1 nucleotide at the 5′ end of the DNA probe starting from the second base from the 5′ end comprise LNA nucleotide(s)

19. The method according to claim 17 or 18, wherein the phosphorothioate bonds and/or LNA nucleotide(s) are included at the 5′ end of the DNA probe starting from the second base from the 5′ end.

20. The method according to any one of the preceding claims, wherein a plurality of stretches of unknown identity are profiled in the same experiment, wherein each stretch of unknown identity is flanked by stretches of known identity, and whereby, for each stretch of unknown identity, at least one DNA probe having a first and a second end is/are provided, which are designed to hybridize to the sequences of known identity flanking the nucleotide stretch of unknown identity, thereby allowing profiling of multiple mutations, insertions, deletions and/or single nucleotide variations.

21. The method according to any one of the preceding claims, wherein if the method is performed in situ, sequencing is performed in situ, such as by Sequencing by Synthesis (SBS), Sequencing by Ligation (SBL), Sequencing by Hybridisation (SBH), SOLiD sequencing or single base extension sequencing.

22. The method according to any one of the preceding claims, wherein the method is performed in vitro, sequencing is performed via NGS sequencing post extraction of DNA probe(s) from the RNA sample.

23. The method according to any one of the preceding claims, wherein if the method is performed in situ, the probes, either pre or post amplified, are extracted from the RNA sample and processed for NGS sequencing.

24. The method according to any one of the preceding claims, wherein the method is used for both in situ and in vitro applications of: mutation profiling, sequencing of insertion or deletions, CRISPR validation, lineage tracing, barcoding applications, miRNA profiling, profiling of unknown sequences in RNA in solution and/or mutation profiling for diagnostics applications.

25. A kit for use in a method for in-situ and/or in vitro profiling of mutations, insertions, deletions and/or single nucleotide variations in an RNA sample according to any one of claims 1 to 24, wherein the RNA sample comprises at least one nucleotide stretch of unknown identity, flanked by nucleotide stretches of known identity, comprising:
(i) one or more DNA probe(s), chosen from (a) at least two linear probes or (b) one or more DNA padlock probes, having a first and a second end, which first end is designed to hybridize to the RNA sample to the at least one nucleotide stretch of known identity at the 3′ side of the nucleotide stretch of unknown identity, and the second end is designed to hybridize to the RNA sample to the nucleotide stretch of known identity at the 5′ side of the nucleotide stretch of unknown identity, optionally designed to be chemically ligated via click reaction;
(ii) a polymerase with reverse transcriptase activity and optionally limited strand displacement, optionally including necessary reagents and buffers;
(iii) a ligase that can use RNA as a splint molecule, optionally including necessary reagents and buffers;
(iv) optionally one or more amplification primers and a polymerase for linear or rolling circle amplification, and necessary reagents and buffers;
(v) optionally means for sequencing the rolling circle amplification product;
(vi) optionally means for library preparation,
(vii) optionally universal bases; and
(iix) instructions for use.

26. The kit according to claim 25, wherein the polymerase with reverse transcriptase activity is chosen from the group comprising BST DNA polymerase (FullLength), TTH DNA Polymerase and DNA Polymerase I, as well as polymerases derived from the thermophilic bacterium Bacillus stearothermophilus, family A of DNA polymerases and other polymerases and reverse transcriptases exhibiting low strand displacement activity.

27. The kit according to claim 25 or 26, wherein the ligase is chosen from Splint R ligase, PBCV-1 DNA ligase and/or Chlorella virus DNA ligase.

28. The kit according to claim 25, further comprising means and reagents for performing the ligation via CuAAC click chemistry.

29. The kit according to any one of claims 25 to 28, wherein the at least one DNA probe(s) are designed to exhibit a gap in the interval of 1-20 nucleotides between the first and second end upon hybridization to the RNA sample and/or wherein the at least one DNA probe are either phosphorylated or unphosphorylated at the 5′ end, and/or wherein the lengths of the first and second ends of the at least one DNA probe that are designed to hybridize to the RNA sample to the nucleotide stretches of known identity are in the interval of 10-30 nucleotides.

30. The kit according to any one of claims 25 to 28, wherein the at least one DNA probe(s) are designed to exhibit a gap in the interval of 1-20 nucleotides between the first and second end upon hybridization to the RNA sample and/or wherein the at least one hybridized DNA probe has an alkyne functional group at the 5′ end, and/or wherein the lengths of the first and second ends of the at least one DNA probe that are designed to hybridize to the RNA sample to the nucleotide stretches of known identity are in the interval of 10-30 nucleotides.

31. The kit according to any one of claim 25 to 30, wherein the means for sequencing the rolling circle amplification product comprises reagents, necessary probes and/or fluorophores for sequencing the at least one amplified circularized oligonucleotide.

32. The kit according to any one of claims 25 to 31, wherein the means for library preparation comprises reagents for preparing a library of profiles of mutations, insertions, deletions and/or single or nucleotide variations in the RNA sample.
